(19) United States
(12) Patent Application Publication
(10) Pub. No.: US 2005/0049434 A1
(43) Pub. Date: Mar. 3, 2005

(54) Fluidizable carbon catalysts

(76) Inventors: Gerald Charles Tustin, Kingsport, TN (US); Joseph Robert Zoeller, Kingsport, TN (US); Richard Dale Colberg, Kingsport, TN (US)

Correspondence Address: ERIC D. MIDDLEMAS;EASTMAN CHEMICAL COMPANY, P. O. BOX 511, KINGSPORT, TN, 37662-5075, US

(21) Appl. No.: 10/650,510

(22) Filed: Aug. 28, 2003

(51) Int. Cl. B01J 31/00; B01J 21/18; C07C 51/10; C07C 51/14

(52) U.S. Cl. 562/517; 502/159; 502/180; 502/182

(57) ABSTRACT

Disclosed are fluidizable catalysts comprising carbonized, polysulfonated vinylaromatic polymer particles. These carbonized polymer particles can be active catalysts by themselves or can act as supports for active catalyst components. These novel catalysts show excellent fluidization behavior over a wide range of gas velocities. Also disclosed are processes for making fluidizable catalysts, for fluidizing these catalysts, and for the preparation of carbonylation products with these catalysts.

### FIELD OF THE INVENTION

This invention pertains to fluidizable catalysts comprising carbonized, polysulfonated vinylaromatic polymer particles. These carbonized polymer particles can be active catalysts by themselves or can act as supports for active catalyst components. This invention further pertains to a process for making such fluidizable catalysts, a process for fluidizing these catalysts, and the use of these catalysts in carbonylation processes.

### BACKGROUND OF THE INVENTION

The use of carbon as a catalyst and a catalyst support material is known. As a support material, carbon offers several advantages over other materials. For example, carbon is much more inert to attack by acid or caustic and has higher thermal stability than silica or alumina. In addition, the inert qualities of the carbon surface in comparison to other supports such as, for example, silica, alumina, or titania, minimizes interaction between the support and the active catalyst component, which may be desirable in some catalyst systems.

Although the surface of carbon is relatively inert compared to those of silica and alumina, carbon can act as a catalyst by itself without the presence of other catalytically active components (see, for example, Bansal et al. in Active Carbon, Marcel Dekker, New York, 1988, pp 413-441). Examples of carbon catalyzed reactions include the oxidation of hydrogen sulfide to sulfur, the reaction of phosgene with formaldehyde to produce dichloromethane and carbon dioxide, and the conversion of hydrogen and bromine to hydrogen bromide. Carbons also are reactive for the conversion of ethylbenzene into styrene as disclosed by Foley et al. in Ind. Eng. Chem. Res., 35 (1996) 3319-3331.

Carbon is a preferred catalyst support for numerous vapor phase reactions. For example, U.S. Pat. No. 4,379,940 discloses zinc supported on carbon as a catalyst for the conversion of acetylene and acetic acid to vinyl acetate. Carbon has been used as a support for metals active for the vapor phase carbonylation of alcohols. For example, U.S. Pat. No. 3,689,533 describes the use of carbon-supported rhodium for the carbonylation of alcohols. U.S. Pat. No. 5,588,143 describes a carbon-supported rhodium catalyst modified by the presence of alkali metal for the carbonylation of methanol. U.S. Pat. No. 5,900,505 describes the use of carbon supported iridium catalysts for the carbonylation of methanol in which carbon is the preferred support. Fujimoto et al. have described nickel on carbon carbonylation catalysts in Journal of Catalysis, 133 (1992) 370-382 and in Chemistry Letters (1987) 895-898.

Many of these vapor phase processes catalyzed by carbon-supported catalysts or by carbon itself are highly exothermic. The exothermic nature of these reactions often makes heat removal difficult which, in turn, often creates reactor control problems, reduces yields, and limits conversions. The heat removal problem can be alleviated by the use of a reactor that is a composite of multiple smaller reactors, although these reactors are very expensive. Reactors containing internal temperature zones can also alleviate the heat removal but they also are expensive. Fluidized bed reactors can provide excellent heat removal and control for many catalytic reactions. Carbon catalysts and support materials, however, are often unsuitable for use within fluidized bed reactors because of poor attrition resistance, poor hardness, and low crush strength.

There have been several approaches to prepare carbon supports and/or catalysts with good attrition resistance, hardness and crush strength. These include the preparation of hybrid catalysts and catalyst supports prepared by coating, spray-drying, or impregnating carbon on an abrasion-resistant inorganic carrier such as silica or alumina (see, for example, U.S. Pat. Nos. 4,206,078; 5,037,791, and 5,072,525). These hybrid catalysts and catalyst supports, however, are difficult and expensive to prepare, and do not provide the chemical resistance of a catalyst or support prepared from carbon alone.

Several carbon catalysts and/or supports exhibiting greater hardness and attrition resistance have been described and exemplified in U.S. Pat. Nos. 4,045,368; 5,569,635; and Japanese Kokai Patent No. Hei 5-163007. These materials, however, either have low surface areas or exhibit particle diameters and bulk densities which result in poor fluidization properties. Carbogenic molecular sieves, described by Foley et al. in Access in Nonporous Materials; Pinnavaia, T. J., Thorpe, M. F., Eds. (1995), can exhibit good hardness and attrition resistance but contain only very small micropores (less than 15 angstroms) and are not well suited for catalytic applications due to the slow rates of diffusion of reactants and products out of the micropore system. Hard, spherical, high surface area carbon catalysts and supports can be manufactured by the pyrolysis of spherical, sulfonated or polysulfonated divinylbenzene-styrene copolymers, available commercially under the trademark Amberlite® 200 (Rohm and Haas Company) as described in U.S. Pat. Nos. 4,040,990; 4,063,912; 4,267,055; and 4,839,331; and European Patent Application No. 0 520 779 A2. The pyrolized resins are available commercially as Ambersorb® adsorbents (Rohm and Haas Company). These carbon catalysts and supports do not exhibit good fluidization properties and often show large bubbles or slugging within the fluidization zone.

The carbon materials described above thus suffer from either poor attrition resistance, low catalytic efficiency, or poor fluidization properties. There is a need, therefore, for a carbon support and/or carbon catalyst that exhibits excellent fluidization properties while retaining high catalytic efficiency, attrition resistance, and mechanical strength.

### SUMMARY OF THE INVENTION

We have discovered that efficient and attrition resistant carbon catalysts or carbon-supported catalysts with excellent fluidization properties may be produced from carbonized polysulfonated vinylaromatic polymer particles having an average particle diameter from about 1 to about 200 micrometers (abbreviated herein as “μm”). Thus, the present invention provides a fluidizable carbon catalyst comprising carbonized polysulfonated vinylaromatic polymer particles in which the particles have an average particle diameter of about 1 to about 200 (μm). More specifically, our invention provides a hard, attrition-resistant, high surface area spherical carbon prepared by the pyrolysis of polysulfonated divinylbenzene-styrene copolymers, which can act as supports for active catalyst components or be active catalysts by themselves. The fluidizable catalysts utilized in the invention have surface areas between 100 and 2000 m²/g, and contain a balance of macropores, mesopores and micropores which enable high rates of chemical reaction. The spherical shape, superior physical properties, and average diameters of the catalysts utilized in the invention result in excellent fluidization behavior.

The carbonized fluidizable polymer particles of our invention also may be used as supports for numerous metal catalysts and catalyst components. Thus, our invention also provides a fluidizable catalyst comprising carbonized polysulfonated vinylaromatic polymer particles and at least one catalyst component selected from alkali metals, alkaline earth metals, metal hydroxides, halides, inorganic acids, and metals from Groups 4-12 of the Periodic Table of the Elements in which the particles have an average particle diameter of about 10 about 130 μm; a BET surface area of about 500 to about 1200 m²/g; and a pore volume ratio of about 0.7 to about 10. When at least one catalyst component is selected from tin and metals from Groups 8-10 of the Periodic Table of the Elements, fluidizable carbonylation catalysts may be obtained. Our invention, therefore, includes a fluidizable carbonylation catalyst comprising carbonized polysulfonated vinylaromatic polymer particles and at least one first metal selected from iron, cobalt, nickel, ruthenium, rhodium, palladium, osmium, iridium, platinum, and tin. The catalyst is useful for the carbonylation of methanol to acetic acid and methyl acetate to acetic anhydride, and ethylene to propionic acid in a fluidized bed under carbonylation conditions of temperature and pressure.

The present invention also provides a process for the preparation of a fluidizable catalyst and for the preparation of a fluidizable carbonylation catalyst comprising carbonized polysulfonated vinylaromatic polymer particles. Further, our invention provides a fluidization process and a process for the preparation of a carbonylation product using the fluidizable carbonylation catalysts of the invention.

### DETAILED DESCRIPTION

The present invention provides a fluidizable catalyst comprising carbonized polysulfonated vinylaromatic polymer particles in which the particles have an average particle diameter of about 1 to about 200 micrometers (μm). The catalysts of our invention provide a hard, attrition-resistant, high surface area, spherical carbon derived from the pyrolysis of polysulfonated divinylbenzene-styrene copolymer particles, which can act as supports for active catalyst components or be active catalysts by themselves. The catalysts utilized in the invention have surface areas between 100 and 2000 m²/g, and contain a balance of macropores, mesopores and micropores allowing for high rates of chemical reaction in a fluidized mode. Also provided is a process for the preparation of and for fluidizing the catalysts of our invention. Our catalysts also may include other catalyst components including alkali metals, alkaline earth metals, metal hydroxides, halides, inorganic acids, and metals from Groups 4-12 of the Periodic Table of the Elements. Our invention also includes fluidizable carbonylation catalysts in which metals or metal compounds useful for carbonylation reactions, such as iron, cobalt, nickel, ruthenium, rhodium, palladium, osmium, iridium, platinum, and tin, are supported on the carbonized, polysulfonated polymer particles, a process for the preparation of these fluidizable carbonylation catalysts, and a process for the preparation of a carbonylation product. The fluidizable catalysts of our invention provide high catalytic efficiency, a high mechanical strength, and a defined particle size distribution and bulk density which make them particularly useful as fluidizable catalysts. Our catalysts, thus, are particularly advantageous for highly exothermic chemical processes, such as, for example, the carbonylation of methanol to acetic acid, where operation in a fluidized bed with the efficient removal of heat from the reaction zone is desirable.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, each numerical parameter should at least be construed in light of the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.1113, etc., and the endpoints 0 and 10. Also, a range associated with chemical substituent groups such as, for example, “C₁ to C₅ hydrocarbons”, is intended to specifically include and disclose C₁ and C₅ hydrocarbons as well as C₂, C₃, and C₄ hydrocarbons.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The catalysts of the present invention are fluidizable. Throughout the specification and the claims, the term “fluidizable”, “fluidization”, or “fluidized” as used herein to describe catalysts or other particulate material, mean catalysts or particles which are capable of being suspended on a moving gas such as, for example, a gas stream that passes through the catalyst or particle bed, causing the suspended particles to behave like a fluid. Although many different sizes and shapes of solid particles may be fluidized, the fluidizable catalysts of the present invention typically have an average particle diameter between about 1 to about 200 micrometers (abbreviated hereinafter as “μm”) and exhibit well behaved, uniform fluidization behavior. Examples of other average particle diameters exhibited by our catalysts include about 5 to about 150 μm and about 10 to about 130 μm. By uniform fluidization behavior, it is meant that the catalyst particles of the invention form a fluidized bed which expands uniformly once a certain minimum fluidization velocity is achieved. The catalyst particles remain in the bed over a wide range of gas velocities. Movement of the vessel containing the fluidized bed results in liquid-like movement of the particles in the bed. Some bubbling is normal and can be minimized by the design of the vessel containing the bed.

By contrast, the carbon catalysts and supports described in the documents cited hereinabove typically have much larger particle diameters and exhibit substantial “spouting” or “slugging” during fluidization. For example, commercially available carbonized polysulfonated polymer particles such as Ambersorb®, typically have a particle size distribution of 150 to about 840 μm. The commercially available carbonized polymer particles show poor fluidization behavior and often exhibit slugging and spouting during fluidization. Spouting occurs where gas channels through most of the bed and exits from a few spots on the top of the bed. Some of the solid sprays upward from the spots where the gas exits and then returns to the bed. There is little increase in superficial gas velocity or the bed height and little expansion of the bed between the onset of fluidization and the onset of slugging. There may be violent movement of the particles in the bed where large bubbles are formed causing the bed to expand and then collapse. During slugging there is movement of the particles in the bed, but it is not uniform. Mass and heat transfer is generally poor under these conditions and becomes worse as the size of the bubbles increases.

Our fluidizable catalysts comprise carbonized, polysulfonated vinylaromatic polymer particles. As used herein, the terms “carbonized, polysulfonated vinylaromatic polymer particles” and intended to be synonymous and used interchangeably with the terms “carbonized polymer particles”, “catalysts”, “catalyst particles”, or “carbonized catalyst particles”. The preparation of the carbonized polysulfonated vinylaromatic polymer particles is described in general in U.S. Pat. No. 4,839,331. The term “carbonized”, as used herein, is intended to be synonymous with the term “pyrolyzed” and refers to polysulfonated vinylaromatic polymer particles which have been substantially transformed to carbon or a carbonaceous material by pyrolysis or the action of heat. The vinylaromatic polymers used in our invention are typically macroporous copolymers and include macroporous or macroreticular copolymers which may be obtained commercially or prepared by suspension polymerization in the presence of a precipitant, as described in U.S. Pat. Nos. 4,256,840 and 4,224,415, and copolymers into which large pores have been introduced by other methods as for example the technique described in U.S. Pat. No. 3,122,514. The resins prepared from macroporous copolymers are called macroporous resins. The term “polysulfonated” or “polysulfonation”, as used herein, refers to a sulfonation process that is sufficiently vigorous to introduce an average of more than one sulfonate group per aromatic nucleus. Such vigorous sulfonation is accompanied by the formation of a significant number of sulfone crosslinks, in which sulfonate groups bridge between two aromatic nuclei to form SO₂ crosslinking groups.

The vinylaromatic polymers of the present invention are those in which at least 50% of the repeating units contain a vinylaromatic group. An example of a vinylaromatic polymer is a polymer in which at least 90% of the repeating units contain a vinylaromatic group. Another example of a vinylaromatic polymer is a polymer in which at least 98% of the repeating units contain a vinylaromatic group. Vinylaromatic monomers include, among others, styrene, alpha-methylstyrene, vinyltoluene, p-methylstyrene, ethyl-vinylbenzene, vinylnaphthalene, divinylbenzene, trivinylbenzene, vinylisopropenylbenzene, diisopropenyl-benzene, and the like. Typically, the monomers used to prepare the vinylaromatic polymers of the present invention are styrene and divinylbenzene (which will normally contain some ethylvinylbenzene).

The polysulfonation reaction is conducted by contacting the vinyaromatic polymer resin with fuming sulfuric acid (oleum) for a period of from about 5 hours to about 20 hours or more at a temperature of about 100° C. to about 150° C. Typically, the polysulfonation reaction is carried out at about 120° C. for a period of about 16 hours. The fuming sulfuric acid may have a specific gravity of about 1.88 to about 2.00 and is used in amounts of from about 100% to about 2000% or more, based on the weight of the vinylaromatic polymer resin. For example, 20% oleum, having a specific gravity of 1.915, may be used at about 1400 to about 1500% of the weight of the vinylaromatic polymer resin. The polysulfonated resin product is typically quenched slowly with water, washed to remove any residual acid, and dried prior to pyrolysis. Care must be taken in the hydration step not to shatter the resin by direct contact with water; hydration with diluted sulfuric acid is preferred.

The pyrolysis may be conducted by methods known to persons skilled in the art, for example, as described in U.S. Pat. No. 4,040,990. For example, the pyrolysis step may be carried out in a controlled manner at temperatures from about 300° C. to about 1200° C. for a period of about 15 minutes to about two hours; in the absence of activating chemicals, the pyrolysis may be maintained longer at the upper temperature with little change taking place in weight loss or pore size development. The polymer may be agitated and/or heated with steam or hot gases or may be heated under static conditions under nitrogen. Because of the small particle diameter of the polymer particles of the present invention, the pyrolysis process is typically performed in the fluidized bed in the presence of an inert gas stream. The flow of inert gas used is generally selected to be the minimum required to fluidize the polysulfonated vinylaromatic copolymer in the carbonization reactor. The carbonized polysulfonated vinylaromatic polymer may be further activated with steam, carbon dioxide, oxygen, carbon monoxide, ammonia and the like as disclosed in U.S. Pat. No. 4,839,331, but this step also is best performed in the fluidized bed mode because of the small particle diameter. When an activating gas is used, the flow is generally near the minimum required for fluidization.

The polymer may be introduced directly into the oven at the highest temperature desired, or may be heated in several steps to the final temperature. As the polysulfonation produces both sulfonate and sulfone groups, analytical identification of the polysulfonated resin is best done by conventional microanalytical procedures for elemental sulfur content. In general, conventional sulfuric acid sulfonation of lightly crosslinked copolymers will introduce approximately the same amount of sulfur as would theoretically be expected for complete monosulfonation of the copolymer. In highly crosslinked copolymers, however, sulfonation tends to occur predominantly at or near the surface of the copolymer particle, and to a lesser extent at increasing distances from the surface. Polysulfonation exhibits a similar phenomenon; a highly crosslinked, polysulfonated copolymer may contain less sulfur than theoretically expected for monosulfonation, yet the accessible aromatic nuclei will be polysulfonated.

Sulfone crosslinking occurs under the same vigorous reaction conditions required to achieve polysulfonation, and is therefore present in polysulfonated resins. The preparation of such resins is described, for example, in U.S. Pat. No. 3,158,583. Besides the two-step sulfonations described in this reference, the copolymers may also be polysulfonated with oleum alone, to obtain a polysulfonated resin operable in the present invention. Other procedures for preparing polysulfonated aromatic cation exchange resins will be apparent to those skilled in the art. Examples of copolymers to be polysulfonated are those prepared by polymerizing a monovinyl aromatic monomer, preferably styrene, and a polyvinyl crosslinking monomer, preferably diisopropenylbenzene or divinylbenzene, to produce macroporous copolymers. Such copolymer particles may be produced in bead form by suspension polymerization and more preferred are those in which a precipitant such as those taught in U.S. Pat. No. 4,256,840 is included in the suspension mixture to produce macroporous polymer beads. Copolymer particles also may be obtained commercially, for example, Amberchrom® CG-300m highly crosslinked 50-100 micron divinylbenzene-styrene spherical beads suspended in ethanol (obtained from Supelco).

The polyvinyl crosslinker level in the copolymer may be from about 2% to about 98% by weight of the copolymer, with the preferred range being from about 3% to about 80% by weight of the copolymer. Suitable crosslinkers include those discussed by Neely in U.S. Pat. No. 4,040,990. Combinations of crosslinkers may also be used.

The carbonized polysulfonated vinylaromatic polymer particles may have any shape but particles in the form of beads or having a rounded or substantially spherical shape are preferred to obtain the best fluidization properties. Rapid hydration of the polysulfonated resins can cause the initially spherical particles to crack and disintegrate. Disintegrated particles are not as well suited for fluidized beds as beads or spheres because of higher attrition rates and poorer fluidization dynamics than spherical particles. If care is taken with the hydration of the initial spherical polysulfonated resins, then the final carbonized products will be spherical since the starting divinylbenzene-styrene copolymers are also spherical.

The particles of the carbonized polysulfonated vinylaromatic polymers of our invention have an average particle diameter of about 1 to about 200 μm. The term “average particle diameter”, as used herein, means the total diameter of all the particles divided by the total number of particles. The average particle diameter of the carbonized polymer particles may be measured by optical microscopy using techniques known to persons skilled in the art. Typically, the microscopy measurement is conducted by measuring the diameters of a small, representative sample of particles containing, typically, 100 to 500 particles, and then calculating the average diameter by dividing the total diameter measurement by the number of particles. The microscopy measurement of particle diameters may be carried out manually or by using automated instrumentation and procedures well known to skilled persons. Very small particles are cohesive and cause the gas to channel through the catalyst bed making fluidization of the bed difficult. When very large particles are fluidized, bubbles tend to form in the bed resulting in poor mass and heat transfer. Preferred average particle diameters are from about 5 to about 150 microns. The most preferred average particle diameters are from about 10 to about 130 microns.

The carbonized polysulfonated vinylaromatic polymer particles utilized in the present invention should have bulk densities from about 0.15 and 1.00 g/cm³. Bulk density is the weight of an assemblage of particles divided by the volume the particles and thus includes the void space between and within the particles. Typical bulk densities are from about 0.20 to about 0.80 g/cm³ and from about 0.25 to about 0.70 g/cm³. When other catalytically active components are added to the carbonized polysulfonated divinylbenzene-styrene copolymers, the bulk density will increase in accord with the amount of material added. The addition of other catalytically active components will not significantly alter the fluidization dynamics other than by requiring an increase in the minimum fluidization velocity.

The carbonized polysulfonated vinylaromatic polymer particles typically have BET surface areas between about 100 and 2000 m²/g. The determination of the surface area by the BET method is well known to persons skilled in the art (see, for example, van Santen et al. Catalysis: An Integrated Approach, 2^{nd} Ed., Amsterdam: Elsevier, 1999, Chapter 13). Materials with low surface areas may have lower catalytic activity but will have higher density and higher attrition resistance. Materials with high surface area may have higher catalytic activity but lower density and attrition resistance. Other examples of BET surface areas are from about 300 to about 1500 m²/g and from about 500 to about 1200 m²/g. The optimal BET surface area will also depend on the nature of the catalytic reaction. The catalytic reaction may require special catalyst porosity characteristics and these will influence the magnitude of the surface area.

The carbonized polysulfonated vinylaromatic polymer particles have a mixture of macropores (pore diameters greater than about 500 angstroms), mesopores (pore diameters between about 20 and 500 angstroms) and micropores (pore diameters less than 20 angstroms). The pore volume ratio, defined as (macopore volume+mesopore volume)/(micropore volume), may be from about 0.5 to about 20. Lower pore volume ratios are favored by reactions that are shape selective and require the constraints imposed by micropores and that have low fouling rates. Higher pore volume ratios are favored by reactions where mass transfer rates can become rate limiting and for reactions that tend to foul the catalyst. Typical pore volume ratios for the catalysts of present invention are from about 0.7 to about 10 and from about 1.0 to about 8.

The carbonized polysulfonated vinylaromatic polymer particles utilized in the present invention must be attrition resistant in order to be useful as catalysts and catalyst supports in fluidized bed reactions. Attrition can be evaluated in a number of different ways, including weight loss after fluidization, crush strength measurements, and grinding. For the catalysts of the present invention, it is convenient to measure the decrease in average particle diameter after the material is fluidized for 5 days in a 15 mm ID glass tube with a nitrogen stream at one atmosphere pressure and ambient temperature with the particle bed volume expanding to between 40 and 50% over the particle bed volume with no gas flow. The average particle diameter after the test should be the same as the before the test within the standard deviation of the two average particle diameter measurements, and the particles should retain their spherical shape.

The fluidizable catalysts of our invention may comprise carbonized poly-sulfonated vinylaromatic polymer particles with or without additional catalyst components. Typically, the fluidizable catalyst comprises carbonized polysulfonated vinylaromatic polymer particles and at least one catalyst component such as, for example, alkali metals, alkaline earth metals, metal hydroxides, metal oxides, halides, inorganic acids, organic halides, and metals from Groups 4-12 of the Periodic Table of the Elements. Examples of additional catalyst components include, but are not limited to, sodium hydroxide, sodium oxide, potassium hydroxide, cesium hydroxide, barium hydroxide, barium oxide, calcium hydroxide, calcium oxide, magnesium oxide, magnesium hydroxide, hydrochloric acid, phosphoric acid, phosphomolybdic acid, sulfuric acid, and metals or metal compounds from Groups 8-12 of the Periodic Table of the Elements such as rhodium, palladium, and iron, and catalytically active metals such as zinc and copper from other regions of the Periodic Table. The additional catalyst components can be incorporated into the carbonized polysulfonated divinylbenzene-styrene copolymer using impregnation techniques well known to those skilled in the art or be used as components of the vapor phase medium. Combinations of these additional components may be used depending on the nature of the reaction being catalyzed.

The fluidizable catalysts comprise carbonized polysulfonated vinylaromatic polymer particles which may have a range of average particle diameters, for example, an average particle diameter is about 1 to about 200 μm, about 5 to about 150 μg/m, and about 10 to about 130 μm. The particles may have a BET surface area of about 100 to about 2000 m²/g; and a pore volume ratio of about 0.5 to about 20. Further examples of other BET surface areas exhibited by the polymer particles are from about 300 to about 1500 m 2/g and from about 500 to about 1200 m²/g. Further examples of pore volume ratios for the polymer particles of present invention are from about 0.7 to about 10 and from about 1.0 to about 8.

The catalysts of the present invention may be fluidized in a flowing or moving gas. Thus, our invention provides a fluidization process comprising providing to a fluidization zone a fluidizable catalyst comprising carbonized polysulfonated vinylaromatic polymer particles in which the particles have an average particle diameter of about 1 to about 200 micrometers (μm) and contacting the catalyst with a gas stream at a superficial gas velocity sufficient to suspend the catalyst in the gas stream. The fluidizable catalyst particles of our process may have a BET surface area of about 300 to about 1500 m²/g and the pore volume ratio is about 0.5 to about 20. Our fluidization process may include removing a portion of the fluidizable catalyst from the fluidization zone for recycling, purging, regeneration, etc., or maintaining the entire charge of catalyst within the reaction zone without any catalyst removal. As used in the present description and in the claims, the term “superficial gas velocity” is defined as the combined volumetric flow rate of vaporized feedstock, including gaseous diluents which can be present in the feedstock, and conversion products, divided by the cross-sectional area of the reaction zone. The superficial gas velocity may be from about 0.002 cm/sec to about 3000 cm/sec. One of skill in the art will understand that the minimum fluidization velocity is the gas velocity required where drag and buoyancy on the particles overcome their weight and any interparticle forces and begin to exhibit fluidization behavior. Persons skilled in the art will also understand that the minimum fluidization velocity is a function of many variables including gas viscosity, the average particle diameter, the particle density and the gas density. The gas properties will in turn be related to the identity of the gas, its temperature and its pressure. Thus the minimum fluidization velocities of the process of the invention can span a large range. The minimum fluidization velocity will be lowest for the smallest lowest density particles in a high-viscosity, high-density gas. The minimum fluidization velocity will be highest for the largest highest density particles in a low-viscosity, low-density gas and may be calculated by methods well known in the art. For example, at 1 bar pressure the minimum fluidization velocities can range from about 0.002 cm/sec to about 0.2 cm/sec for particles with an average diameter of 1 micron depending on the particle and gas densities and gas viscosity. At 1 bar pressure the minimum fluidization velocities can range from about 0.02 cm/sec to about 20 cm/sec for particles with an average diameter of 100 microns depending on the particle and gas densities and gas viscosity. At 1 bar pressure the minimum fluidization velocities can range from about 0.003 cm/sec to about 0.5 cm/sec for particles with an average diameter of 5 microns depending on the particle and gas densities and gas viscosity. At 1 bar pressure the minimum fluidization velocities can range from about 0.1 cm/sec to about 60 cm/sec for particles with an average diameter of 200 microns depending on the particle and gas densities and gas viscosity. At 1 bar pressure the minimum fluidization velocities can range from about 0.004 cm/sec to about 0.7 cm/sec for particles with an average diameter of 10 microns depending on the particle and gas densities and gas viscosity. At 1 bar pressure the minimum fluidization velocities can range from about 0.04 cm/sec to about 30 cm/sec for particles with an average diameter of 130 microns depending on the particle and gas densities and gas viscosity. The preceding minimum fluidization velocity ranges are mathematical estimates only and values outside these ranges may be possible. The best way to establish the minimum fluidization velocity is to measure it experimentally. The mathematical estimates are useful for selecting the initial regions of actual experimentation.

The process of the invention can be conducted over a wide range of gas velocities. The lowest velocities are those described above as minimum fluidization velocities. Gas flow rates can be increased considerably above the minimum fluidization velocity while maintaining most of the particles within the fluidized bed. The single-particle terminal velocity is the gas velocity required to maintain a single particle suspended in an upwardly flowing vapor stream. The single-particle terminal velocity is also a function of many variables including gas viscosity, the particle diameter, the particle density and the gas density. The gas properties will in turn be related to the identity of the gas, its temperature and its pressure. Thus the single-particle terminal velocities of the process of the invention also can span a very large range. For example, at 1 bar pressure, the single-particle terminal velocities can range from about 0.3 cm/sec to about 50 cm/sec for particles with an average diameter of 1 micron depending on the particle and gas densities and gas viscosity. In another example, at 1 bar pressure, the single-particle terminal velocities can range from about 2 cm/sec to about 500 cm/sec for particles with an average diameter of 100 microns depending on the particle and gas densities and gas viscosity. In yet another example, at 1 bar pressure, the single-particle terminal velocities can range from about 0.3 cm/sec to about 150 cm/sec for particles with an average diameter of 5 micron depending on the particle and gas densities and gas viscosity. In a further example, at 1 bar pressure, the single-particle terminal velocities can range from about 5 cm/sec to about 1200 cm/sec for particles with an average diameter of 200 microns depending on the particle and gas densities and gas viscosity. In yet another example, at 1 bar pressure, the single-particle terminal velocities can range from about 0.5 cm/sec to about 200 cm/sec for particles with an average diameter of 10 micron depending on the particle and gas densities and gas viscosity. In yet another example, at 1 bar pressure, the single-particle terminal velocities can range from about 3 cm/sec to about 800 cm/sec for particles with an average diameter of 130 microns depending on the particle and gas densities and gas viscosity. The preceding single-particle terminal velocity ranges are mathematical estimates only and values outside these ranges may be possible. The best way to establish the single-particle terminal velocity is to measure it experimentally. The mathematical estimates are useful for selecting the initial regions of actual experimentation.

The gas velocity where particles are removed from a bed containing an assemblage of particles can be 10 to 100 times higher than the single particle terminal velocity due to interactions among the particles. The velocity of the gas may be such that particles are actually removed from the reaction zone, and this may actually be advantageous if the catalyst needs to be regenerated outside of the reaction zone. Particles that have been removed from the reactor may be returned, discarded, or replaced with new particles.

Our fluidizable catalysts are prepared by sulfonating the corresponding vinylaromatic polymer particles with a sulfonating reagent under sulfonation conditions and pyrolyzing the sulfonated polymer by heating at elevated temperatures. Thus, an embodiment of the present invention is a process for preparation of a fluidizable catalyst comprising: (i) contacting vinylaromatic polymer particles having an average particle diameter of about 1 to about 200 μm in a reaction zone with 30% oleum under sulfonation conditions of time, temperature, and pressure to produce a reaction mixture comprising polysulfonated vinylaromatic polymer particles; (ii) washing the polysulfonated vinylaromatic polymer particles from step (i) with water; and (iii) heating the polysulfonated vinylaromatic polymer particles from step (ii) at a temperature from about 600° C. to about 1000° C. The activity of fluidization catalysts may be increased by further contacting the carbonized polysulfonated vinylaromatic polymer particles with steam or other surface area enhancing reactants such as, for example, CO₂, oxygen, air, or ammonia. The process of our invention, therefore, further comprises contacting the carbonized polysulfonated vinylaromatic polymer particles from step (iii) with steam, oxygen, carbon dioxide, air, or ammonia at a temperature from about 700° C. to about 1000° C. In another embodiment of our process for the preparation of a fluidizable catalyst, the vinylaromatic polymer particles of step i) have a average particle diameter of about 5 to about 150 μm; a BET surface area of about 300 to about 1500 m²/g; and a pore volume ratio of about 0.5 to about 20. In yet another embodiment, the vinylaromatic polymer particles of step i) have a average particle diameter of about 10 to about 130 μm; a BET surface area of about 500 to about 1200 m²/g; and a pore volume ratio of about 1.0 to about 8.

Because the fluidizable catalysts of the instant invention may incorporate a large variety of catalyst components, the number and range of chemical reactions which may be catalyzed by our catalysts is also large. Examples of reactions which may be catalyzed by the catalysts of the invention include, but are not limited to, hydrogenation reactions, dehydrogenation reactions, oligomerization reactions, olefin metathesis reactions, oxidation reactions, elimination reactions, addition reactions, nucleophilic substitution reactions, electrophilic substitution reactions, carbonylation, and decarbonylation reactions. The main requirements are that the reaction is thermodynamically feasible under the reaction conditions, that the catalyst facilitates the desired transformation, and that the reactants and products are in the vapor phase. The process is particularly well suited for those reactions that are best performed in the presence of carbon-based catalysts. These reactions include, but are not limited to, hydrodesulfurization over Co—Mo on carbon, the synthesis of alcohols from carbon monoxide and hydrogen over Mo—K on carbon, the synthesis of vinyl acetate from acetylene and acetic acid over Zn or Hg on carbon, the reaction of hydrogen fluoride with chlorinated or chlorofluorinated organic molecules over chromium on carbon, the hydroformylation (i.e., hydrocarbonylation) of olefins in the presence of carbon monoxide and hydrogen, and the carbonylation of alcohols, ethers and esters in the presence of halide over Group VIII metals on carbon.

The fluidizable catalysts can be used over a large temperature range that depends on the nature of the reaction being performed. The main requirements are that the temperature be such that the reaction is conducted in the vapor phase under conditions where it is thermodynamically feasible and where the catalyst has reasonable thermal stability. Because the range of possible reactions is large, process temperatures may range from about −180° C. to about 1400° C. Most reactions will occur between about −100 and 1000° C. or more commonly between about 0 and 800° C.

Similarly, the fluidizable catalysts may be used over a large range of pressures depending on the nature of the reaction being performed. The main requirement is that the pressure be sufficiently low to keep the reactants and products in the vapor phase at the temperature of the reaction. The range of possible reactions is also large; thus, process pressures may range from about 0.01 bar absolute (bara) to about 10000 bara. More common pressure ranges will be between 0.1 and 1000 bara with the most common range being between about 1 bara and 100 bara.

Our fluidizable catalysts are particularly useful as carbonylation catalysts. The term “carbonylation”, as used herein, means a chemical reaction or process where a carbonyl radical is introduced into a molecule, typically by the insertion of carbon monoxide into one or more chemical bonds of a reactant or reaction intermediate. Non-limiting examples of carbonylation reactions are the reaction of methanol with carbon monoxide to give acetic acid, the reaction to methyl acetate with carbon monoxide to give acetic anhydride, and the hydroformylation reaction of ethylene with carbon monoxide and hydrogen to give propionaldehyde. The term “hydroformylation”, as used herein, is synonymous with “hydrocarbonylation” and “oxo reaction” and means the reaction of an ethylenically unsaturated compound with carbon monoxide and gaseous hydrogen to produce an aldehyde or an oxygenated product derived from an aldehyde. Thus, one embodiment of our invention is a fluidizable carbonylation catalyst comprising carbonized polysulfonated vinylaromatic polymer particles and at least one first metal selected from iron, cobalt, nickel, ruthenium, rhodium, palladium, osmium, iridium, platinum, and tin in which the particles have a average particle diameter of about 1 to about 200 μm; a BET surface area of about 500 to about 1200 m²/g; and a pore volume ratio of about 1.0 to about 8. Our fluidizable carbonylation catalysts may also comprise particles having other average particle diameters such as, for example, an average particle diameter of about 5 to about 150 μm and about 10 to about 130 μm. Optionally, the catalyst may also include a second metal, selected from an alkali, an alkaline earth, lanthanides, gold, mercury, and transition metals selected from the group vanadium, niobium, tantalum, titanium, zirconium, hafnium, molybdenum, tungsten, and rhenium, and combinations thereof. Preferably, the first metal is rhodium or iridium. In another embodiment of our invention, the fluidizable carbonylation catalyst is not a hydroformylation catalyst.

The compound or form of the first metal(s) used to prepare the catalyst is not critical and may be selected from such complexes as halides, acetates, nitrates, acetonylacetates, and mixtures thereof. For example, when iridium or rhodium is the active metal, the catalyst may be prepared from any of a wide variety of iridium or rhodium containing compounds containing a myriad of combinations of halide, trivalent nitrogen, organic compounds of trivalent phosphorous, carbon monoxide, hydrogen, and 2,4-pentane-dione, either alone or in combination. Such materials are available commercially and may be used in the preparation of the catalysts utilized in the present invention. In addition, the oxides of iridium or rhodium may be used if dissolved in the appropriate medium. Typically, rhodium or iridium are employed as a salt of one of its chlorides such as, for example, iridium trichloride or rhodium trichloride or hydrated trichlorides, hexacholoro-iridate and any of the various salts of hexachloroiridate(IV). One skilled in the art will understand that use of the iridium and rhodium complexes or other Group VIII and tin metals should be comparable on the basis of cost, solubility, and performance.

The compound or form of the second metal generally is not critical, and may be any of a wide variety of compounds containing one or more of the secondary metals. For example, when metals from the Lanthanide Series are used, they may be present either alone or in combination. A wide variety of compounds of these elements containing various combinations of halides, acetates, nitrates, cyclopentadiene, and 2,4-pentane-dione, either alone or in combination, are available commercially and may be used in the preparation of the catalysts utilized in the process of the present invention, including naturally occurring blends of the Lanthanides. In addition, the oxides of these materials may be used if dissolved in the appropriate medium. Desirably, the compound used to provide the second metal is a water soluble form of the metal(s). Preferred sources include acetates, nitrates, and their halides. The selection of these salts is dictated by solubility, preferably water solubility, which can vary widely across this list of useful second components. Additional examples of second metals which may be used include lanthanum, cerium, praseodymium, and neodymium, or combinations thereof. The halides of these secondary metals are generally commercially available and water soluble. Still further examples of second metal are samarium, europium, gadolinium, terbium, dysprosium, holmium, or erbium and mixtures of thereof.

The amount of the first metal and any second metal catalyst component can each vary from about 0.01 weight % (abbreviated herein as “wt %”) to about 10 wt % based on the total weight of the fluidizable catalyst. Further examples of amounts for the first and second metal components are from about 0.05 wt % to about 5 wt % and from about 0.1 wt % to about 2 wt % of each metal component being more preferred, wherein the aforementioned wt % is based on the total weight of the fluidizable catalyst.

In addition to the metal catalyst components, the fluidizable carbonylation catalyst, optionally, may also comprise at least one halogen promoter selected from iodine, bromine, and chlorine which may also be catalytically active and which aids in the carbonylation process. The halogen promoter is normally included as a metal halide. Examples of metal halides which may be used are sodium iodide, lithium iodide, and potassium iodide.

The fluidizable carbonylation catalysts are prepared by contacting the carbonized polysulfonated vinylaromatic polymer particles with a solution of the metal catalyst components. Our invention thus provides a fluidizable carbonylation catalyst prepared by a process comprising:
i) providing carbonized polysulfonated vinylaromatic polymer particles having
   an average particle diameter of about 1 to about 200 μm;
   a particle BET surface area of about 100 to about 2000 m²/g; and
   a pore volume ratio of about 0.5 to about 20;
ii) contacting the particles in step (i) with a solution comprising from about 0.01 wt % to about 20 wt %, based on the total weight of the solution, of at least one first metal selected from iron, cobalt, nickel, ruthenium, rhodium, palladium, osmium, iridium, platinum, and tin;
iii) drying the particles from step (ii);
   Optionally, one or more second metals may be included by (iv) contacting the dried particles of step (iii) with a solution comprising from about 0.01 wt % to about 20 wt %, based on the total weight of the solution, of at least one second metal selected from alkali metals, alkaline earth metals, lanthanide metals, gold, mercury, vanadium, niobium, tantalum, titanium, zirconium, hafnium, molybdenum, tungsten, and rhenium and (v) drying the particles. In addition to a first and second metal component, the fluidizable carbonylation catalyst optionally may include a halogen promoter. Our invention, therefore, also provides a fluidizable carbonylation catalyst prepared by further including the optional steps of (vi) contacting the dried polymer particles from step (iii) or step (v) above with a solution comprising from about 0.01 wt % to about 20 wt %, based on the total weight of the solution, of a metal halide selected from sodium iodide, lithium iodide, or potassium iodide; and vii) drying the particles from step (vi). The catalyst particles, either before or after any of the impregnation steps described above, optionally may be activated by contacting the dried catalyst particles with steam, oxygen, carbon dioxide, air, or ammonia at a temperature from about 700° C. to about 1000° C.

The contacting of a first metal and, if so employed, a second metal and/or halogen promoter, with the carbonized polymer particles is carried out by preferably dissolving or dispersing the metal components and halogen promoter in a suitable solvent to form a solution, dispersion, or suspension. Typically, the liquid used to deliver the catalyst components, e.g., the first and second metals and halogen promoter, will have a boiling point, or a high vapor pressure (e.g., from about 600 mm to about 760 mm) at a temperature of from about 10° C. to about 140° C. Examples of solvents include carbon tetrachloride, benzene, acetone, methanol, ethanol, isopropanol, isobutanol, pentane, hexane, cyclohexane, heptane, toluene, pyridine, diethylamine, acetaldehyde, acetic acid, tetrahydrofuran and water. The solid support material is then contacted and desirably impregnated with the metal containing solutions. Various methods of contacting the support material with catalyst components may be employed. For example, an iridium containing solution can be admixed with a second metal solution prior to impregnating the support material. Alternatively, the respective solutions can be impregnated separately into or associated with the carbonized polymer particles sequentially. The order of impregnation or deposited of the first and second metal components and the halogen promoter is not important. Drying the catalyst particles before any impregnation or deposition step is desirable but not critical. For example, the catalyst components may be associated with the support material in a variety of forms such as slurries which can be contacted with the carbonized polymer particles in a trickle bed column. Alternatively, the carbonized polymer particles may be immersed in excess solutions of the active components with the excess being subsequently removed using techniques known to those skilled in the art. The solvent or liquid is evaporated and the catalyst particles are dried so that at least a portion of the catalyst components is associated with the carbonized polymer particles. Drying temperatures may range from about 100° C. to about 600° C. One skilled in the art will understand that the drying time is dependent upon the temperature, humidity, and solvent. Generally, lower temperatures require longer heating periods to effectively evaporate the solvent from the catalyst particles.

Impregnation is only one means for associating the various catalyst components with the solid support matrix. Other suitable methods for contacting the catalyst components with the carbonized polymer particles include sublimation and plasma deposition. These and other alternative methods of preparation, are familiar to practitioners of the art.

The fluidizable carbonylation catalysts of the instant invention may be used to prepare a carbonylation product. Thus, our invention provides a process for the preparation of a carbonylation product comprising: (1) feeding a gaseous mixture comprising carbon monoxide, a carbonylatable reactant, and a halide selected from chlorine, bromine, iodine and compounds thereof to a carbonylation zone which (i) contains a fluidizable carbonylation catalyst comprising carbonized polysulfonated vinylaromatic polymer particles and at least one first metal selected from iron, cobalt, nickel, ruthenium, rhodium, palladium, osmium, iridium, platinum, and tin in which the particles have a average particle diameter of about 1 to about 200 μm; (ii) is maintained under carbonylation conditions of temperature and pressure; and (2) recovering a gaseous effluent comprising a carbonylation product from the carbonylation zone. The gaseous mixture of step (1) is fed to the carbonylation zone at a superficial gas velocity sufficient to suspend the carbonylation catalyst in the gaseous mixture. The term “carbonylation product”, as used herein, is intended to mean one or more organic compounds produced by the insertion of carbon monoxide into one or more chemical bonds of a reactant or reaction intermediate. Typical carbonylation products are carboxylic acids, esters, aldehydes, and anhydrides. The carbonylation product of the present invention is not intended to be limited to a single product, but may include multiple products. For example, the process of the invention converts alcohols into carboxylic acids and esters. In absence of halides, olefins may be hydroformylated in the presence of carbon monoxide and hydrogen to aldehydes. Another embodiment of our invention, however, does not include the preparation of hydroformylation products. Olefinic alcohols my be converted in lactones. In the substantial absence of water, ethers are converted into carboxylic esters and anhydrides. In the presence of sufficient water, ethers are converted into carboxylic acids and esters. In the substantial absence of water, esters are converted into carboxylic acid anhydrides. In the presence of sufficient water, esters are carbonylated to give carboxylic acids, which may react with any alcohols that are present (from hydrolysis of the starting ester) to produce additional esters. Examples of carbonylation products are acetic acid, methyl acetate, acetic anhydride, or mixtures thereof.

The term “carbonylatable reactant”, as used herein, refers to one or more organic compounds capable of reacting with carbon monoxide, under carbonylation conditions of temperature and pressure, to produce a carbonylation product resulting from the insertion of carbon monoxide into one or more chemical bonds of the reactant or an reaction intermediate produced from the reactant. Carbonylatable reactants include, but are not limited to, olefins which may be converted to aldehydes or oxygenated derivatives of aldehydes by hydroformylation; alkyl alcohols and their derivatives, including ethers, esters and mixtures of the same; alkyl, alkenyl, aryl, aralkyl, and heteroaryl halides; and olefins that can react with water, alcohols, hydrogen halides, aliphatic acids or carboxylic acids also present in the reactant stream under carbonylation process conditions to produce alcohols, alkyl halides, ethers, esters, or alcohol derivatives in situ. Non-limiting examples of carbonylatable reactants include alcohols and ethers in which an aliphatic carbon atom is directly bonded to an oxygen atom of either an alcoholic hydroxyl group in the compound or an ether oxygen in the compound and may further include aromatic moieties. The feedstock may comprise one or more lower alkyl alcohols having from 1 to 10 carbon atoms, alkane polyols having 2 to 6 carbon atoms, alkyl alkylene polyethers having 3 to 20 carbon atoms and alkoxyalkanols having from 3 to 10 carbon atoms. The carbonylatable reactant may comprise methanol, ethanol, methyl acetate, dimethyl ether, and mixtures thereof. The most preferred carbonylatable reactant is methanol. Although methanol is preferably used in the process and is normally fed as methanol, it can be supplied in the form of a combination of materials which generate methanol in situ. Examples of such combination of materials include (i) methyl acetate and water and (ii) dimethyl ether and water. In the operation of the process, both methyl acetate and dimethyl ether are formed within the reaction zone and, unless methyl acetate is the desired product, these products may be recycled with water to the reaction zone where they are later consumed to form acetic acid. Thus, one skilled in the art will recognize that it is possible to utilize the present invention to produce carboxylic acid from a corresponding ester feed material.

Although the presence of water in the gaseous feed mixture is not essential when using methanol, the presence of some water is desirable to suppress formation of methyl acetate and/or dimethyl ether. When using methanol to generate acetic acid, the molar ratio of water to methanol can be about 0:1 to about 10:1, but preferably is about 0.01:1 to about 1:1. When using an alternative source of methanol such as methyl acetate or dimethyl ether, the amount of water fed usually is increased to account for the mole of water required for hydrolysis of the methanol alternative. Therefore, when using either methyl acetate or dimethyl ether, the mole ratio of water to ester or ether is about 1:1 to about 10:1, but preferably is about 1:1 to about 3:1. In the preparation of acetic acid, it is apparent that combinations of methanol, methyl ester, and/or dimethyl ether are equivalent, provided the appropriate amount of water is added to hydrolyze the ether or ester to provide the methanol reactant. When the process is operated to produce methyl acetate, preferably no water should be added and dimethyl ether becomes the preferred feedstock. Further, when methanol is used as the feedstock in the preparation of methyl acetate, it is preferable to remove water.

The carbon monoxide may be fed to the carbonylation zone either as purified carbon monoxide or as carbon monoxide including other gases. The carbon monoxide need not be of high purity and may contain from about 1% by volume to about 99% by volume carbon monoxide, and preferably from about 70% by volume to about 99% by volume carbon monoxide. The remainder of the gas mixture may include such gases as nitrogen, hydrogen, water and paraffinic hydrocarbons having from one to four carbon atoms. Although hydrogen is not part of the reaction stoichiometry, hydrogen may be useful in maintaining optimal catalyst activity. Therefore, the preferred ratio of carbon monoxide to hydrogen is about 99:1 to about 2:1, but ranges with even higher hydrogen levels are also useful. The amount of carbon monoxide useful for the carbonylation reaction ranges from a molar ratio of about 0.1:1 to about 1,000:1 of carbon monoxide to alcohol, ether or ester equivalents with a more preferred range being from about 0.5:1 to about 100:1 and a most preferred range from about 1.0:1 to about 20:1.

The process of this invention is operated in the vapor phase and, therefore, is practiced at temperatures above the dew point of the carbonylation product mixture. However, since the dew point is a complex function of dilution (particularly with respect to non-condensable gases such as unreacted carbon monoxide, hydrogen, or inert diluent gas), product composition, and pressure, the process may still be operated over a wide range of temperatures, provided the temperature exceeds the dew point of the product effluent. The term “dew point”, as used herein, means the temperature, at a given pressure, at which a gas is saturated with respect to its condensable components and at which condensation occurs. The dew point of the carbonylation products of the present invention may be calculated by methods well known to those skilled in the art, for example, as described in Perry's Chemical Engineer's Handbook, 6^{th} ed, (McGraw-Hill), pp. 13-25 through 13-126. Dew points for single product or complex mixtures may be calculated using commercially available engineering computer programs, such as Aspen®, also well-known to those skilled in the art. In practice, the process typically operates at a temperature range of 100 to 250° C. Other examples of temperature ranges in which our process may operate include 120 to 240° C. and 150 to 240° C.

As with temperature, the pressure range is dependent, in part, upon the dew point of the product mixture. However, provided that the reaction is operated at a temperature sufficient to prevent liquefaction of the product effluent, a wide range of pressures may be used, e.g., pressures of about 0.1 to about 100 bars absolute (bara). The process preferably is carried out at a pressure of about 1 to about 50 bara and, most preferably, about 3 to about 30 bara.

The process of the invention employs a halide selected from chlorine, bromine and iodine compounds. Preferably, the halide is selected from bromine and iodine compounds that are vaporous under vapor phase carbonylation conditions of temperature and pressure. Suitable halides include hydrogen halides such as hydrogen iodide and gaseous hydroiodic acid; alkyl and aryl halides having up to about 12 carbon atoms such as methyl iodide, ethyl iodide, 1-iodopropane, 2-iodobutane, 1-iodobutane, methyl bromide, ethyl bromide, benzyl iodide and mixtures thereof. Desirably, the halide is a hydrogen halide or an alkyl halide having up to about 6 carbon atoms. Non-limiting examples of preferred halides include hydrogen iodide, methyl iodide, hydrogen bromide, methyl bromide and mixtures thereof. The halide may also be a molecular halogen such as I₂, Br₂ or Cl₂. The most preferred halide is iodide. Non-limiting examples of the most preferred vaporous halides include methyl iodide, hydrogen iodide and molecular iodine. The amount of vaporous halide present typically ranges from a molar ratio of about 1:1 to about 10,000:1 of alcohol, ether or ester equivalents to halide, with the preferred range being from about 5:1 to about 1000:1. In one embodiment of the invention, the halide is selected from iodine, hydrogen iodide and methyl iodide and the carbonylation zone is maintained at a temperature of about 100 to 350° C. and a pressure of about 1 to 50 bar absolute.

Our process utilizes a fluidizable carbonylation catalyst comprising carbonized polysulfonated vinylaromatic polymer particles and at least one first metal selected from iron, cobalt, nickel, ruthenium, rhodium, palladium, osmium, iridium, platinum, and tin in which the particles have a average particle diameter of about 1 to about 200 μm. Other examples of average particle diameters which may be exhibited by the carbonized polymer particles are about 5 to about 150 μm and about 10 to about 130 μm. The catalyst may have a BET surface area of about 500 to about 1200 m²/g and a pore volume ratio of about 1.0 to about 8.

The fluidizable carbonylation catalyst of our process may further comprise at least one halogen promoter selected from iodine, bromine, and chlorine which may also be catalytically active and which aids in the carbonylation process. The halogen promoter is normally included as a metal halide. Examples of metal halides which may be useds are sodium iodide, lithium iodide, and potassium iodide. Optionally, the catalyst may also include one or more second metals, selected from an alkali, an alkaline earth, lanthanides, gold, mercury, and transition metals selected from vanadium, niobium, tantalum, titanium, zirconium, hafnium, molybdenum, tungsten, and rhenium. Preferably, the first metal is rhodium or iridium.

The amount of the first metal and any second metal catalyst component can each vary from about 0.01 wt % to about 10 wt % based on the total weight of the fluidizable catalyst. Further examples of the amounts of the first and second metal components are from about 0.05 wt % to about 5 wt % and from about 0.1 wt % to about 2 wt % of each metal component being more preferred, wherein the aforementioned wt % is based on the total weight of the fluidizable catalyst. The compound or form of the first and optional second metal components used to prepare the catalyst is not critical and may be selected from such complexes as halides, acetates, nitrates, acetonylacetates, and mixtures thereof as described hereinabove.

Our process is useful for the preparation of acetic acid, methyl acetate, or a mixture thereof. The present invention, therefore, provides a process for the preparation of acetic acid, methyl acetate, or a mixture thereof comprising:
(1) feeding a gaseous mixture comprising carbon monoxide, methanol, and a halide selected from iodine, hydrogen iodide, and methyl iodide to a carbonylation zone which (i) contains a fluidizable carbonylation catalyst comprising carbonized polysulfonated vinylaromatic polymer particles, rhodium, and lithium iodide in which the particles have a average particle diameter of about 1 to about 200 μm; (ii) is maintained at a temperature of about 150 to 275° C. and a pressure of about 3 to 50 bar absolute; and
(2) recovering a gaseous product comprising acetic acid from the carbonylation zone. The gaseous mixture of step (1) is fed to the carbonylation zone at a superficial gas velocity sufficient to suspend the carbonylation catalyst in the gaseous mixture. Further examples of average particle diameters which the carbonized polymer particles may have are about 5 to about 150 μm and about 10 to about 130 μm. The fluidizable carbonylation catalyst may have a BET surface area of about 500 to about 1200 m²/g; and a pore volume ratio of about 1.0 to about 8. The gaseous mixture may contain water in an amount which gives a water:methanol mole ratio of about 0.01:1 to 1:1. In another embodiment, the carbonylation zone contains a fluidizable carbonylation catalyst prepared as described hereinabove.

As noted hereinabove, our novel fluidizable carbonylation catalysts may be used for the preparation of a hydroformylation product. Thus, our invention also includes a process for the preparation of a hydroformylation product comprising:
(1) feeding a gaseous mixture comprising carbon monoxide, hydrogen, and an olefin to a hydroformylation zone which (i) contains a fluidizable carbonylation catalyst comprising carbonized polysulfonated vinylaromatic polymer particles and at least one metal selected from iron, cobalt, nickel, ruthenium, rhodium, palladium, osmium, iridium, platinum, and tin in which the particles have a average particle diameter of about 1 to about 200 μg/m; (ii) is maintained under hydroformylation conditions of temperature and pressure; and
(2) recovering a gaseous effluent comprising a hydroformylation product from the hydroformylation zone;
   in which the gaseous mixture of step (1) is fed to the hydroformylation zone at a superficial gas velocity sufficient to suspend the carbonylation catalyst in the gaseous mixture. Further examples of average particle diameters which the carbonized polymer particles may have are about 5 to about 150 μm and about 10 to about 130 μm. The fluidizable carbonylation catalyst may have a BET surface area of about 500 to about 1200 m²/g; and a pore volume ratio of about 1.0 to about 8. In another embodiment, the hydroformylation zone contains a fluidizable carbonylation catalyst prepared as described hereinabove.

The term “hydroformylation product”, as used herein, is intended to mean one or more organic compounds produced by the reaction of an ethylenically unsaturated compound with carbon monoxide and gaseous hydrogen to produce an aldehyde or an oxygenated product derived from an aldehyde. Thus, typically, hydroformylation products are aldehydes but may include compounds resulting from the further reaction of the initial aldehyde products under hydroformylation conditions of temperature and pressure, such as hydrogenation to give alcohols, i.e., “oxo alcohols”, reaction to give aldol condensation products, and Tischenko reactions to give alcohols and esters. The olefins that may be hydroformylated by means of our process comprise aliphatic, alicyclic, aromatic and heterocyclic mono-, di- and tri-olefins containing up to 10 carbon atoms. Examples of the aliphatic olefins that may be utilized in the process include straight- and branched-chain, unsubstituted and substituted, aliphatic mono-α-olefins containing up to 10 carbon atoms. Examples of the groups that may be present on the substituted mono-α-olefins include hydroxy; alkoxy including ethers and acetals; alkanoyloxy such as acetoxy; amino including substituted amino; carboxy; alkoxycarbonyl; carboxamido; keto; cyano; and the like.

Mixtures of olefins also can be used in the practice of this invention. The mixtures may be of the same carbon number such as mixtures of n-octenes or it may represent refinery distillation cuts which will contain a mixture of olefins over a range of several carbon numbers. The olefin reactants which are particularly preferred comprise mono-α-olefins of 2 to 10 carbon atoms, especially propylene.

The reaction conditions used are not critical for the operation of the process and conventional hydroformylation conditions normally are used. The process requires that an olefin is contacted with hydrogen and carbon monoxide in the presence of the novel catalyst system described hereinabove. While the process may be carried out at temperatures in the range of 20 to 200° C., the preferred hydroformylation reaction temperatures are from 50 to 150° C. with the most favored reaction temperatures ranging from 80 to 130° C.

The hydroformylation process of the present invention normally is carried out at elevated pressures in the range of 0.7 to 69 bars gauge (barg; 10 to 1000 pounds per square inch-psig), preferably in the range of 6.9 to 27.6 barg (about 100 to 400 psig). Lower pressures result in the rate of reaction being economically unattractive whereas higher pressures, e.g., greater than 69 barg, result in increased gas compression and equipment costs. In the present invention, the synthesis gas, i.e., CO and H₂, is introduced into the reactor in a continuous manner by means, for example, of a compressor. The partial pressures of the ratio of the hydrogen to carbon monoxide in the feed is selected according to the desired linear to branched isomer ratio in the product. Generally, the partial pressure of hydrogen and carbon monoxide in the reactor is maintained within the range of 0.4 to 13 barg (about 5 to 188 psig) for each gas. The partial pressure of carbon monoxide in the reactor is maintained within the range of 0.4 to 13 barg (about 5 to 188 psig) and is varied independently of the hydrogen partial pressure.

The molar ratio of hydrogen to carbon monoxide can be varied widely within these partial pressure ranges for the hydrogen and carbon monoxide. The ratios of the hydrogen to carbon monoxide and the partial pressure of each in the synthesis gas can be readily changed by the addition of either hydrogen or carbon monoxide to the synthesis gas stream. For example, the hydrogen:carbon monoxide mole ratio in the reactor may vary from 10:1 to 1:10.

The amount of olefin present in the vapor phase also is not critical. In the hydroformylation of a gaseous olefin feedstock such as propylene, the partial pressures in the vapor space in the reactor typically are in the range of 0.01 to 34 barg. In practice the rate of reaction is favored by high concentrations of olefin in the vapor phase. In the hydroformylation of propylene, the partial pressure of propylene preferably is greater than 0.4 barg, e.g., from 0.4 to 9 barg. In the case of ethylene hydroformylation, the preferred partial pressure of ethylene in the reactor is greater than 0.01 barg.

The present invention is illustrated by the following examples.

### EXAMPLES

### Example 1

This example describes the fluidization behavior of Rh-Li on carbonized polysulfonated divinylbenzene-styrene copolymer at one atmosphere pressure. A carbonized polysulfonated divinylbenzene-styrene copolymer was prepared from two 100 mL samples of Amberchrom® CG-300m highly crosslinked 50-100 micron divinylbenzene-styrene spherical beads suspended in ethanol obtained from Supelco. The mixture was filtered and the wet solids heated on the steam bath under vacuum to yield the dried polymer (55.7 g). The divinylbenzene-styrene beads had a surface area equal to 700 m²/gram, an average pore size of 300 angstroms, a porosity of 55-75 volume percent and a skeletal density of 1.05 g/cc. The dried polymer was transferred to a one-liter three-necked flask fitted with an overhead stirrer, condenser, nitrogen inlet and a thermowell. The thermowell contained a thermocouple from a temperature controller used to measure the temperature of the contents of the flask and to control the temperature. Thirty percent oleum (d=1.925 g/mL, 782 g) was added to the flask under a nitrogen atmosphere, and the mixture was heated to 125° C. over a 5-hour period and maintained at 125° for an additional 16 hours. The temperature was lowered to 100° C. and water (125 mL) was added over a period of 2.5 hours. The temperature was then lowered to 90° C. and additional water (250 mL) was added over a period of 4 hours. The mixture was cooled to ambient temperature. A portion (about 150 mL) of the liquid was decanted and additional water was added (250 mL), the mixture exotherming 10° C. as the water was slowly added. The decantation water addition process was continued until no additional exotherm was seen upon the water addition. The mixture was filtered and washed with water (8 L) until the washings were colorless and then with methanol (3×500 mL). The resulting wet solid was transferred to a one-liter flask and dried on the steam bath under vacuum over the weekend to yield the dried polysulfonated divinylbenzene-styrene copolymer (110.1 g). The dried polysulfonated divinylbenzene-styrene copolymer was divided into two equal portions, and one portion was loaded into a 25 mm OD (22 mm ID) quartz tube containing a quartz wool plug to support the polysulfonated material. The quartz tube was positioned vertically in a Lindberg three element electric tube furnace having a 24 inch long heated zone, and the column of polysulfonated material was 16.5 inches high. Nitrogen was delivered to the base of the quartz tube via a Tylan model FC-260 mass flow controller at a rate of 20 standard cubic centimeters per minute (SCCM) causing the bed to fluidize and increase in height another 0.75 inches (1.9 cm). A second quartz wool plug was placed at the top of the heated zone in the quartz tube to prevent the solids from leaving the reactor. The material was heated with the 20 SCCM nitrogen flow over a period of one hour to 800° C. and held at 800° C. for 30 minutes in the flowing nitrogen and then cooled to ambient temperature. The carbonized material was removed from the quartz tube, and the pyrolysis procedure was repeated on the second portion of dried polysulfonated divinylbenzene-styrene copolymer. The two batches of carbonized material were combined to give a total yield of 38.82 g black particles. Optical microscopy determined that the spherical particles had an average particle diameter of 61.12 microns with a standard deviation of 11.02 with particle sizes ranging between 42 and 87 microns. Surface area analysis was performed using a Micromeritics model ASP 2010 surface area analyzer, and the material had a BET surface area of 628 m²/g and an average pore diameter of 46.4 angstroms. The volume of pores less than 661 angstroms was 0.729 cm³/g and that less then 3000 angstroms was 0.945 cm³/g. The carbonized material had a bulk density of 0.31 g/mL.

A portion of the carbonized material (36.36 g) was further activated by steam. The carbonized material was loaded into the same quartz tube used previously and was supported by a quartz wool plug. The quartz tube was placed vertically into the Lindberg three element electric tube furnace, and an additional quartz wool plug was placed at the top of the heated zone. An electrically heated adapter for feeding nitrogen and water from a syringe was fitted to the base of the reactor and heated to 140° C. A second nitrogen inlet with oil bubbler at the top of the reactor was used to insure that the system was always under a positive pressure of nitrogen. Nitrogen (20 SCCM) was flowed through the base of the apparatus overnight, and then the furnace was heated to 900° C. over a period of one hour. The base nitrogen was turned off and water was fed to the heated base using a Harvard Apparatus syringe pump at a rate of 2.16 mL/hr. The process was continued until 17 mL water had been delivered from the syringe pump, and then the mixture was allowed to cool under a flow of nitrogen (20 SCCM) from the reactor base. The steam-activated material isolated from the quartz tube (21.57 g) had a bulk density of 0.26 g/mL. The average particle diameter measured by optical microscopy was 55.95 microns with a standard deviation of 8.09, and the particle sizes ranged from 40 to 84 microns. The steam-activated catalyst had a BET surface area of 1137 m²/g and an average pore diameter of 39.4 angstroms. The volume of pores less than 648 angstroms was 1.12 cm³/g and that less then 3000 angstroms was 1.43 cm³/g.

A portion of the steam-activated catalyst (20.17 g) was placed in an evaporating dish, and a solution was prepared from rhodium trichloride hydrate containing 38.9 wt % Rh (830 mg, 3.14 mmoles) and water (70 mL). The aqueous rhodium solution was poured onto the steam-activated catalyst in the evaporating dish. The mixture was stirred until uniform, and then the mixture was evaporated on the steam bath with occasional stirring until the solids became free flowing. The Rh-impregnated steam-activated catalyst was then transferred to the previously used quartz tube containing a quartz wool support plug. The quartz tube was placed into the Lindberg three element electric furnace and heated in an upward flow of nitrogen (20 SCCM) over a 2 hour period to 300° C. and held at 300° C. for 2 hours before cooling back to ambient temperature. A portion of the dried The Rh-impregnated steam-activated catalyst (14.1 g) was then transferred to an evaporating dish and impregnated with a solution prepared from lithium iodide (1.175 g, 8.78 mmoles) and water (50 mL). The mixture was stirred until uniform then dried on the steam bath until the solids became free flowing. The LiI-Rh-impregnated steam-activated catalyst was then transferred to the previously used quartz tube containing a quartz wool support plug. The quartz tube was placed into the Lindberg three element electric furnace and heated in an upward flow of nitrogen (20 SCCM) over a 1 hour period to 130° C., held at 130° C. for 2 hours, heated to 300° C. over a 1 hour period and held at 300° C. for 2 hours before cooling back to ambient temperature. The dried LiI-Rh-impregnated steam-activated catalyst was recovered from the reactor as black spherical particles (14.92 g).

A glass reactor was used to evaluate the fluidization behavior of the LiI-Rh-impregnated steam-activated catalyst and its reactivity and heat transfer characteristics in the carbonylation of methanol at one atmosphere pressure. The design of the reactor allowed for the direction of the gas flow to be reversed without otherwise interrupting the reaction conditions. This design allowed the reactor to be operated in the fluidized bed mode or in the plug flow mode by turning two stopcocks. The reactor consisted of two major sections. Section A contained the catalyst charge. The base of section A was a 15 mm ID (18 mm OD) glass tube opened at the bottom. A coarse glass frit was located 0.5 inch (1.27 cm) up from the bottom and served as the support element for the catalyst and gas dispersion device for the fluidized bed. The 15 mm ID tube extended upward beyond the glass frit an additional 5.5 inches (14 cm) and then expanded into a sphere with an inner diameter of 25 mm. The spherical region acted as an expansion zone to capture particles that were entrained out of the fluidized bed. The top of the spherical region was open and connected to a 10 mm ID (12 mm OD) tube which continued to extend upward an additional 5 inches and was open at the top. Tube A1 of approximately 2 mm ID (3 mm OD) exited the side of the 10 mm ID tube at 0.4 inch (1 cm) above the top of the spherical region and angled downward below the base of section A. Tube A1 was the tube through which the gases leaving the fluidized bed zone exited section A. After tube A1 was below the base of section A, it expanded to 6 mm ID (8 mm OD) and connected to one of the parallel stems of a double oblique bore three way stopcock S1. Tube A2 (6 mm ID, 8 mm OD) exited the side of the 10 mm ID tube of section A at 3 inches (1.6 cm) above tube A1 and angled upward and behind the 10 mm ID tube of section A. Tube A2 connected one of the parallel stems of a second double oblique bore three way stopcock S2. Tube A2 was used to supply reactant to the catalyst zone when the reactor was operated in the plug flow mode. The top of the 10 mm ID tube of section A was located 2 inches above tube A2 and was open (end A). Catalyst was loaded through end A, and then end A was plugged with a rubber septum. A 0.0625 inch (1.59 mm) OD stainless steel thermowell extended through the rubber septum and continued down to the glass frit. The thermowell was sealed on the bottom and opened on the top to allow for insertion and movement of a thermocouple to record the temperature at various locations inside section A. Section B was glass tubing that encased section A from the region above stopcock S1 and extended to above tube A2. Tubes A1 and A2 passed through the walls of section B, and end A also was outside of section B. Tube B1 extended from the base of section B and joined the remaining parallel stem of the double oblique bore stopcock S1. Reactor effluent exited the reactor through tube B1 when the reactor was operated in the plug flow mode. Tube B2 extended from the wall of section B near the top of the apparatus and angled up and behind the open end A and joined the remaining parallel stem of the double oblique bore stopcock S2. Reactant flowed through tube B2 when the reactor was operated in the fluidized bed mode. Thus when stopcocks S1 and S2 were in the fluidized bed mode, reactant entered through the opening stem of S2, passed through tube B2 into the space between sections A and B, up through the frit into the catalyst bed and out of tube A1 and through stopcock S1 and out the remaining stem of S1. When stopcocks S1 and S2 were in the plug flow mode, reactant entered through the remaining stem of S2, passed through tube A2 through the catalyst bed and frit entering the region between sections A and B and exiting through B1 and through stopcock S1 and out the remaining stem of S1. The dimensions of the reactor allowed the region of the reactor below Tubes A2 and B2 to fit into a vertically mounted Lindberg single element tube furnace having a heated zone 1.75 inches (4.44 cm) in diameter and 12 inches (30.5 cm) long. The heated zone of the furnace extended to the base of section B. The furnace could be opened during operation of the reactor to allow measurement of the height of the fluidized bed.

The reactor was loaded with the dried LiI-Rh-impregnated steam-activated catalyst (10 mL, 2.75 g) and placed into the single element furnace. The height of the catalyst bed with no gas flowing was 52 mm. Nitrogen was metered using a Tylan model FC-260 mass flow controller and the catalyst was fluidized at various bed temperatures and flow rates. The corresponding bed heights are summarized in Table 1.

TABLE 1

| Bed Temperature, ° C. | SCCM N₂ | Bed height, mm |
|---|---|---|
| 23 | 20 | 65 |
| 23 | 41 | 73 |
| 23 | 63 | 76 |
| 23 | 84 | 76 |
| 23 | 105 | 76 |
| 23 | 127 | 76 |
| 162 | 20 | 76 |
| 162 | 41 | 86 |
| 162 | 63 | 84 |
| 162 | 84 | 84 |
| 162 | 105 | 85 |
| 162 | 127 | 85 |
| 207 | 20 | 77 |
| 207 | 41 | 87 |
| 207 | 63 | 87 |
| 207 | 84 | 87 |
| 207 | 105 | 87 |
| 207 | 127 | 87 |
| 230 | 20 | 81 |
| 230 | 41 | 90 |
| 230 | 63 | 90 |
| 230 | 84 | 89 |
| 230 | 105 | 89 |
| 230 | 127 | 89 |

The temperature of the catalyst bed was recorded at 10 mm intervals beginning at the base of the catalyst and extending up to the top of the bed at 90 mm under the conditions of 127 SCCM N2 and 230° C. The temperature at the base of the bed was 231° C. The temperature in the region of 10 to 60 mm was 232° C., and the temperature form 70 to 90 mm was 233° C. Thus the example illustrates the excellent fluidized bed dimensional stability and isothermal temperature profiles that can be achieved with the carbonized polysulfonated divinylbenzene-styrene copolymers utilized in the invention.

### Comparative Example 1

Fluidization behavior of Commercial Carbonized Polysulfonated Polymer Particles. A 13 mm OD glass tube (˜10.6 mm ID) containing a coarse glass frit was positioned vertically and was loaded with 0.9430 grams of Ambersorb 572. The bed height with no gas flowing upward through the tube was 25 mm. Gas (nitrogen and nitrogen+air at high flow rate) was passed upward at ambient temperature and pressure until movement of the bed was observed. No movement of the bed was observed until 250 SCCM flow was reached. Once movement was observed, the following observations were made.

| SCCM Gas | Bed Height, mm | Fluidization Behavior |
|---|---|---|
| 250 | 25 | Slight movement at top of bed |
| 300 | 27 | Slugging |
| 350 | 27-30 | Slugging |
| 400 | 33-35 | Slugging |
| 500 | 33-35 | Bubbles in bed |

Note that the volume of the Ambersorb 572 used in this experiment was about 2 cc. This means that the gas hourly space velocity requited to achieve fluidization in this vessel was 7500 hr⁻¹.

This experiment was performed on the fluidizable catalyst prepared in Example 1 after the pyrolysis (but before the steam activation) described in Example 1. The same vertical 25 mm OD (22 mm ID) quartz pyrolysis tube containing the quartz wool plug described in Example 1 was used in this experiment. The tube contained 19.68 g of the carbonized resin and had a bed height of 6.25 inches with no gas flowing. Nitrogen was passed upward through the bed at ambient temperature and pressure, and the following observations were made.

| SCCM Gas | Bed Height, inches | Remarks |
|---|---|---|
| 20 | 6.5 | No spouting, bubbling or slugging |
| 40 | 7 | Occasional spray |
| 80 | 7.5 | Continuous spray |
| 120 | 7.5 | |
| 160 | 7.5 | No entrainment |
| 200 | 7.75 | |
| 240 | 8 | Some entrainment |

This example shows that a low superficial velocity was needed to fluidize the catalyst particles.

This example illustrates the process of the invention for the carbonylation of methanol at one atmosphere pressure. The carbon monoxide feed to the reactor was provided by a Tylan model FC-260 mass flow controller. The liquid feed to the reactor was 70 wt % methanol/30 wt % methyl iodide and had a density=1.0 g/mL. The liquid feed was delivered to the reactor through a vaporization zone by a DraChrom Series II liquid chromatography pump. The reactor effluent was condensed first at ambient temperature and then at −78° C. The combined condensed products were weighed and analyzed by gas chromatography using a Hewlett Packard Model 6890 gas chromatograph fitted with a 30 m×0.25 mm DB-FFAP capillary column (0.25 micron film thickness) programmed at 40° C. for 5 minutes, 25° C./minute to 240° C. and holding at 240° C. for 1 minute using a thermal conductivity detector held at 250° C. (injector temperature=250° C.). Mixtures were prepared for gas chromatographic analysis by adding 5 mL of tetrahydrofuran solution containing 2 wt % decane internal standard to an accurately weighed 1 gram sample of the product mixture.

The reactor from Example 1 was used in this example. The reactor was loaded with the LiI-Rh-impregnated steam-activated catalyst of Example 1 (10 mL, 2.76 g) and placed into the single element furnace used in Example 1. Carbon monoxide (46 SCCM) was fed to the reactor in the fluidized bed mode, and the furnace temperature was brought to 210° C. The temperature at the base of the catalyst bed was 205° C. after the furnace temperature had stabilized. The 70 wt % methanol/30 wt % methyl iodide was then fed to the reactor at a rate of 0.05 mL/minute. After 1 hour 55 minutes, the furnace was opened briefly, and the bed height was measured at 70 mm. The reaction was continued and monitored until the catalyst activity had increased to a steady value, and during this time he reaction was performed in both the fluidized bed mode and the plug flow mode during this period by turning stopcocks S1 and S2. After 24 hours the catalyst activity had stabilized and a direct comparison was made between the bed temperature profiles and carbonylation activity for both the fluidized bed and the plug flow modes of operation. In Example 3A the reaction was performed in the fluidized bed mode with the furnace set at 210° C., and the average catalyst bed temperature was 215° C. (average of 70 mm bed height at 10 mm intervals). In Example 3B the reaction was performed in the plug flow mode with the furnace set at 210° C., and the average catalyst bed temperature was 223° C. (average of 50 mm bed height at 10 mm intervals). In Example 3C, the reaction was performed in the fluidized bed mode with the furnace temperature increased to 217° C. to bring the average bed temperature to 223° C. Thus, Example 3C allowed for the performance of the fluidized bed mode to be compared to that of the plug flow mode Example 3B at the same average catalyst bed temperature. The catalyst bed temperature profiles are summarized in Table 2.

TABLE 2

| Distance up from | Example 3A | Example 3B | Example 3C |
|---|---|---|---|
| bed base, mm | T_{bed}, ° C. | T_{bed}, ° C. | T_{bed}, ° C. |
| 0 | 214 | 214 | 221 |
| 10 | 216 | 222 | 223 |
| 20 | 215 | 227 | 223 |
| 30 | 216 | 227 | 223 |
| 40 | 216 | 228 | 223 |
| 50 | 215 | 217 | 223 |
| 60 | 216 | Out of bed | 223 |
| 70 | 214 | Out of bed | 221 |

Table 3 summarizes the methanol conversions and acetyl production rates, in moles per liter-hour, for Examples 3A, 3B and 3C. The rates are calculated based on the volume of catalyst charged.

TABLE 3

| | % Methanol | Moles acetic | Moles methyl | Moles total |
|---|---|---|---|---|
| Example | conversion | acid/L-hr | acetate/L-hr | acetyl/L-hr |
| 3A | 49.8 | 1.15 | 0.72 | 1.87 |
| 3B | 57.3 | 1.46 | 0.82 | 2.29 |
| 3C | 55.0 | 0.99 | 1.04 | 2.03 |

Thus, Example 3 illustrates that the fluidized bed process of the invention provides isothermal catalyst bed temperatures and superior heat removal compared to conventional plug flow operation. The example also illustrates that when the average temperatures of the fluidized bed and the plug flow bed are comparable, the methanol conversion and acetyl space-time yield become comparable.

### Example 4

This example illustrates the carbonylation of methanol at elevated pressure. These conditions provide commercially acceptable rates and conversions under isothermal operating conditions. The reactor was constructed entirely of Hastelloy C® alloy. Reactants entered the base of the reactor via a 0.375 inch (9.5 mm) outer diameter (O.D.) inlet tube having a wall thickness of 0.065 inch (1.65 mm). The portion above the inlet tube expanded as a collar piece as a cone into a cylindrical section having a 0.625-inch (1.6 cm) inner diameter (I.D.) and a wall thickness of 0.1875 inch (4.8 mm) with overall length of 2.00 inches (5.1 cm). The top 0.38-inch (9.6 mm) portion of the collar was machined to a diameter of 0.750 inch (1.9 cm). The machined portion of the collar contained a 0.735-inch (1.87 cm) diameter by 0.0625-inch (1.65 mm) thick Hastelloy C® alloy 5 micron metal filter, which acted as a gas dispersion device and support for catalyst. The filter and the collar containing the filter were welded to a 6.25-inch (15.9 cm) long by 0.625-inch (1.6 cm) I.D./0.750-inch ((1.9 cm) O.D. Hastelloy C® alloy reaction tube. The reaction tube was welded to an expanded zone increasing in a conical fashion at 45 degrees to an outer diameter of 1.50 inches (3.81 cm), continuing in a cylindrical fashion for another 1.83 inches (4.56 cm) and then decreasing at a 45-degree angle and welded to a 4.50 inch (11.4 cm) long by 0.375-inch (0.95 cm) O.D. loading and sensing tube. The vertical loading and sensing tube contained a 0.375-inch (0.95 cm) O.D. pressure transducer side arm located 2.0 inches (5.1 cm) above the expanded zone and positioned at 45 degrees from vertical of the loading and sensing tube. Vapor product was removed from the expanded zone through a 0.125 inch (3.18 mm) O.D. product removal line which extended up to approximately half the vertical distance of the expanded zone and off to one side. A Hastelloy C® alloy 5 micron sintered metal filter was welded to the top end of the product removal line. The product removal line exited the expanded zone through the bottom conical portion of the expansion zone and continued downward to a distance past the base of the reactor inlet line.

Metered gas flows were maintained by Brooks 5850 Series E mass flow controllers interfaced with a Camile® 3300 Process Monitoring and Control System. Temperature control was also provided by the Camile® 3300 Process Monitoring and Control System. Liquid feed was provided by an Alltech 301 HPLC pump. Liquid and gas feeds were fed to a heated Hastelloy C® alloy vaporizer maintained at 230° C. and transported through a transfer line at 230° C. to the base of the reactor inlet tube. Heat to the reactor was provided by three separate split aluminum blocks with each split aluminum block surrounded by band heaters. Each split aluminum block heating unit had its own temperature control provided by the Camile® 3300 Process Monitoring and Control System. The bottom heater provided heat to the reactor inlet tube and collar piece. The central heater provided heat to the reaction tube section. The top heated provided heat to the expansion zone. A Hastelloy C® alloy thermowell extending from the top of the reactor to the gas dispersion frit allowed for monitoring the catalyst temperature at various locations inside the reactor.

The end of the product removal line was connected to a Hastelloy C® alloy condenser, which was attached to a Hastelloy C® alloy product collection tank with a working capacity of one liter. The pressure was maintained using a Tescom Model 44-2300 backpressure regulator attached to a vent line on the top of product collection tank. Liquid samples were collected from a valve at the base of the liquid collection tank. Liquid products from the collection tank were weighed and analyzed by gas chromatography as per Example 3.

The reactor was loaded with the LiI-Rh-impregnated steam-activated catalyst of Example 1 (10 mL, 2.62 g). Carbon monoxide was fed to the reactor base at 300 SCCM, and the reactor was pressurized to 200 psig (13.8 barg) while heating to 220° C. Then the 70 wt % methanol/30 wt % methyl iodide liquid feed was fed to the reactor at 0.22 mL/minute while maintaining the carbon monoxide feed at 300 SCCM. The reactor was operated in this fluidized bed mode for 68 hours and 5 product samples were collected during this time. The liquid feed was then stopped and the reactor was allowed to cool under a positive pressure of carbon monoxide. The pressure was then released, the reactor opened and 16×24 mesh quartz chips (25 mL) were loaded on top of the catalyst. The weight of the quartz chips allowed for the catalyst to be evaluated as a packed bed. Carbon monoxide again was fed to the reactor base at 300 SCCM, and the reactor was pressurized to 200 psig (13.8 barg) while heating to 220° C. Then the 70 wt % methanol/30 wt % methyl iodide liquid feed again was fed to the reactor at 0.22 mL/minute while maintaining the carbon monoxide feed at 300 SCCM. The reactor was operated in this packed bed mode for 90 hours and 7 product samples were collected during this time. Table 4 provides the temperature profile of the reactor in the region of the catalyst bed for both the fluidized bed mode and the packed bed mode.

TABLE 4

| Distance up from bed | Fluidized bed | Packed bed temperature, |
|---|---|---|
| base, inches (cm) | temperature, ° C. | ° C. |
| 0 (0) | 235.9 | 269.7 |
| 0.5 (1.3) | 236.2 | 275.8 |
| 1.0 (2.5) | 236.2 | 246.0 |
| 1.5 (3.8) | 237.2 | 231.0 |
| 2.0 (5.1) | 237.3 | 225.8 |
| 2.5 (6.4) | 231.6 | 224.9 |

The height of the fluidized bed was approximately 2 inches (5.1 cm), and the bed was isothermal with a 1.4° C. range over 2 inches (5.1 cm). However, the packed bed exhibited a large temperature gradient with a 50° C. range over 2 inches (5.1 cm), and the average temperature across the 2-inch range was about 250° C. The highest temperature recorded in the packed bed was 323° C. and was recorded 16 hours after the data in Table 4 were collected.

The activity of the catalyst declined faster when the reaction was operated in the packed bed mode. The performance of the catalyst (moles/liter-hour) in both modes of operation as a function of time is shown in Table 5.

TABLE 5

| | Hours | | Moles | Moles | |
|---|---|---|---|---|---|
| | on | % methanol | acetic | methyl | Moles total |
| Mode | stream | conversion | acid/L-hr | acetate/L-hr | acetyl/L-hr |
| Fluid | 23 | 99.5 | 18.4 | 4.0 | 22.4 |
| Fluid | 39 | 99.0 | 19.3 | 4.5 | 23.8 |
| Fluid | 47 | 97.2 | 19.8 | 4.6 | 24.4 |
| Fluid | 63 | 95.6 | 19.6 | 4.4 | 24.0 |
| Fluid | 68 | 95.0 | 19.3 | 3.9 | 23.2 |
| Packed | 86 | 100 | 24.6 | 0.3 | 24.9 |
| Packed | 95 | 100 | 26.3 | 0.3 | 26.6 |
| Packed | 110 | 100 | 26.5 | 0.6 | 27.1 |
| Packed | 119 | 98.8 | 21.9 | 2.8 | 24.7 |
| Packed | 134 | 91.6 | 13.9 | 5.7 | 19.6 |
| Packed | 143 | 91.1 | 14.0 | 5.3 | 19.3 |
| Packed | 158 | 89.4 | 13.8 | 5.6 | 19.4 |

Initially, while at elevated average temperature, the packed bed provided slightly higher rates and conversions than the fluidized bed. However, with increasing time on stream, the catalyst temperature in the packed bed eventually began to fall as the catalyst deactivated. Thus the average methanol conversion decreased 0.174% per hour during operation in the packed bed mode compared to 0.106% per hour during operation in the fluidized bed mode. The average total acetyl production rate decreased 0.116 mole acetyl/L-hr per hour during operation in the packed bed mode compared to an actual slight average rate increase of 0.020 mole acetyl/L-hr per hour during operation in the fluidized bed mode. The average amount of Rh found in the liquid samples collected during the fluidized bed operation was 89 parts per billion (ppb) whereas an average of 155 ppb was found in the samples collected during the packed bed operation. Thus the example amply illustrates that the process of the invention can extend catalyst life by facilitating heat control in the exothermic methanol carbonylation reaction.

### Example 5

This example illustrates the attrition resistance of the catalysts utilized in the invention under fluidized bed conditions. A portion of the steam-activated carbonized polysulfonated resin from Example 1 (0.73 g) was placed in the glass reactor described in Example 1. The average particle diameter of the steam-activated carbonized polysulfonated resin from Example 1 was 55.95 microns with a standard deviation of 8.09 as determined by optical microscopy. The sample was fluidized in nitrogen (40 SCCM) at ambient temperature, and the height of the bed increased from 14 mm to 20 mm during the fluidization. The fluidization was continued for 5 days. The particles were then removed from the reactor and again analyzed by optical microscopy. The average particle diameter measured after the fluidization was 63.41 microns with a standard deviation of 8.25. Thus the average diameter of the particles after the fluidization was essentially the same as before the fluidization within the standard deviation of the measurements. The optical micrographs also indicated that the particles had retained their spherical shape after the fluidization.

### Example 6

This prophetic example illustrates the use of the fluidizable catalysts of the present invention for the oxidative dehydrogenation of ethylbenzene to styrene at one atmosphere pressure. The alternating fluidized bed/plug flow reactor from Examples 1 and 3 is used in this example, liquid feed is provided by a Harvard Apparatus Model 22 syringe infusion pump, and air feed is provided by a Tylan Model FC-260 mass flow controller. Condensed products are combined and weighed as per Example 3 and analyzed by proton nuclear magnetic resonance spectroscopy.

The carbonized polysulfonated divinylbenzene-styrene copolymer particles are prepared as per Example 1, but is not impregnated with any metals after the steam activation step. The reactor is loaded with the steam-activated catalyst (10 mL, 2.75 g), fitted with a thermowell and traveling thermocouple as per Example 1, and heated with the furnace set for 350° C. in 106 SCCM air in the fluidized bed mode. Liquid ethylbenzene is then added to the air stream at 0.071 mL/minute using the syringe pump. The reactor is operated in this mode for 3 hours with an average bed temperature of 365° C. with temperatures spanning the range 364 to 366° C. throughout the 96 mm high fluidized catalyst bed. After 3 hours of operation in the fluidized bed mode, the condensed products are collected. Styrene is produced in 97% selectivity at a rate of 2.0 moles/L-hr at 60% ethylbenzene conversion. The mode of operation is then changed from fluidized bed to plug flow without otherwise altering the conditions. The reactor is operated in this mode for 3 hours with an average bed temperature of 372° C. with temperatures spanning 264-280° C. throughout the 50 mm packed bed. After 3 hours of operation in the plug flow mode, the condensed products are collected, the liquid feed is stopped, and the reactor cooled in 106 SCCM air flow. Styrene is produced in 92% selectivity at a rate of 2.2 moles/L-hr at 66% ethylbenzene conversion. It should be noted that excessive heating of carbon catalysts in the presence of oxygen can cause the catalyst to lose mass because of partial combustion of the catalyst.

### Example 7

This prophetic example illustrates the use of the fluidizable catalysts of the present invention for the reaction of acetic acid with acetylene at one atmosphere pressure. The alternating fluidized bed/plug flow reactor from Examples 1 and 3 is used in this example, liquid feed is provided by a Harvard Apparatus Model 22 syringe infusion pump, and acetylene and nitrogen feeds are provided by Tylan Model FC-260 mass flow controller. A few crystals of tert-butylhydroquinone are added to the condensation train to inhibit the polymerization of the vinyl acetate product. Condensed products are combined and weighed as per Example 3 and analyzed by gas chromatography using a Hewlett-Packard Model 5890 gas chromatograph with flame ionization detection and a 25 m×0.53 mm FFAP capillary column (1.0 micron film thickness) programmed at 40° C. for 5 minutes, 15° C. to 235° C. and holding at 235° C. for 1.67 minutes.

The carbonized polysulfonated divinylbenzene-styrene copolymer particles are prepared as per Example 1 through the steam activation step. A portion of the steam-activated carbon particles (10 g) is placed in an evaporating dish, and a solution is prepared from zinc acetate dihydrate (2.5 g) and water (35 mL). The aqueous zinc solution is poured onto the steam-activated carbon particles in the evaporating dish. The mixture is stirred until uniform, and then the mixture is evaporated on the steam bath with occasional stirring until the solids become free flowing. The zinc impregnated steam-activated catalyst is then transferred to a quartz tube containing a quartz wool plug. The quartz tube is placed into the Lindberg three element electric furnace and heated in an upward flow of nitrogen (20 SCCM) over a 2 hour period to 250° C. and held at 250° C. for 2 hours before cooling back to ambient temperature.

The reactor is loaded with the zinc impregnated steam-activated catalyst (10 mL, 3.33 g), fitted with a thermowell and traveling thermocouple as per Example 1, and heated with the furnace set for 185° C. in 20 SCCM nitrogen in the fluidized bed mode. Liquid acetic acid is then added to the nitrogen stream at 0.028 mL/minute using the syringe pump, and acetylene is fed at 27 SCCM. The nitrogen flow is terminated after the acetylene flow is started. The reactor is operated in this mode for 3 hours with an average bed temperature of 200° C. with temperatures spanning the range 199 to 201° C. throughout the 87 mm high fluidized catalyst bed. After 3 hours of operation in the fluidized bed mode, the condensed products are collected. Vinyl acetate is produced in 99% selectivity from acetic acid at a rate of 2.3 moles/L-hr at 80% acetic acid conversion. The mode of operation is then changed from fluidized bed to plug flow without otherwise altering the conditions. The reactor is operated in this mode for 3 hours with an average bed temperature of 211° C. with temperatures spanning 199-215° C. throughout the 50 mm packed bed. After 3 hours of operation in the plug flow mode, the condensed products are collected, the liquid and acetylene feeds are stopped, and the reactor cooled in 20 SCCM nitrogen flow. Vinyl acetate is produced in 95% selectivity from acetic acid at a rate of 2.4 moles/L-hr at 85% acetic acid conversion. This example again illustrates that the fluidized catalysts of the invention provides isothermal catalyst bed temperatures and superior heat removal compared to conventional plug flow operation.

1. A fluidizable catalyst comprising carbonized polysulfonated vinylaromatic polymer particles in which the particles have an average particle diameter of about 1 to about 200 micrometers (μm).

2. The fluidizable catalyst as recited in claim 1 in which the particles are beads or spheres.

3. The fluidizable catalyst as recited in claim 2 in which the particles have an average particle diameter of about 5 to about 150 μm.

4. The fluidizable catalyst as recited in claim 3 in which the particles have a BET surface area of about 100 to about 2000 m²/g.

5. The fluidizable catalyst as recited in claim 4 in which the particles have a BET surface area of about 300 to about 1500 m²/g and a pore volume ratio of about 0.5 to about 20.

6. A fluidizable catalyst comprising carbonized polysulfonated vinylaromatic polymer particles and at least one catalyst component selected from alkali metals, alkaline earth metals, metal oxides, metal hydroxides, halides, inorganic acids, and metals from Groups 4-12 of the Periodic Table of the Elements in which the particles have
an average particle diameter of about 10 to about 130 μm;
a BET surface area of about 500 to about 1200 m²/g; and
a pore volume ratio of about 0.7 to about 10.

7. The fluidizable catalyst as recited in claim 6 in which the catalyst component comprises at least one compound selected from sodium hydroxide, sodium oxide, potassium hydroxide, cesium hydroxide, barium hydroxide, barium oxide, calcium hydroxide, calcium oxide, magnesium oxide, magnesium hydroxide, hydrochloric acid, phosphoric acid, phosphomolybdic acid, or sulfuric acid.

8. The fluidizable catalyst as recited in claim 6 in which the catalyst component is one or more metals from Groups 8-12 of the Periodic Table of the Elements.

9. A fluidization process comprising i) providing to a fluidization zone a fluidizable catalyst comprising carbonized polysulfonated vinylaromatic polymer particles in which the particles have an average particle diameter of about 1 to about 200 μm and ii) contacting the catalyst with a gas stream at a superficial gas velocity sufficient to suspend the catalyst in the gas stream.

10. The process as recited in claim 9 in which the particles have a BET surface area of about 300 to about 1500 m²/g and a pore volume ratio of about 0.5 to about 20.

11. The process as recited in claim 10 in which the superficial gas velocity is from about 0.002 cm/sec to about 3000 cm/sec.

12. The process as recited in claim 11 further comprising iii) removing a portion of the fluidizable catalyst from the fluidization zone.

13. A process for the preparation of a fluidizable catalyst comprising:
i) contacting vinylaromatic polymer particles having an average particle diameter of about 1 to about 200 μm in a reaction zone with 30% oleum under sulfonation conditions of time, temperature, and pressure to produce a reaction mixture comprising polysulfonated vinylaromatic polymer particles;
ii) washing the polysulfonated vinylaromatic polymer particles from step (i) with water; and
iii) heating the polysulfonated vinylaromatic polymer particles from step (ii) at a temperature from about 600° C. to about 1000° C.

14. The process as recited in claim 13 further comprising:
iv) contacting the polysulfonated vinylaromatic polymer particles from step (iii) with steam, oxygen, carbon dioxide, air, or ammonia at a temperature from about 700° C. to about 1000° C.

15. The process as recited in claim 14 in which the vinylaromatic polymer particles of step i) have
an average particle diameter of about 10 to about 130 μm;
a BET surface area of about 500 to about 1200 m²/g; and
a pore volume ratio of about 1.0 to about 8.

16. A fluidizable carbonylation catalyst comprising carbonized polysulfonated vinylaromatic polymer particles and at least one first metal selected from iron, cobalt, nickel, ruthenium, rhodium, palladium, osmium, iridium, platinum, and tin in which the particles have
a average particle diameter of about 1 to about 200 μm;
a BET surface area of about 500 to about 1200 m²/g; and
a pore volume ratio of about 1.0 to about 8.

17. The fluidizable carbonylation catalyst as recited in claim 16 in which the first metal is rhodium or iridium.

18. The fluidizable carbonylation catalyst as recited in claim 17 further comprising at least one second metal selected from alkali metals, an alkaline earth metals, lanthanide metals, gold, mercury, vanadium, niobium, tantalum, titanium, zirconium, hafnium, molybdenum, tungsten, and rhenium.

19. The fluidizable carbonylation catalyst as recited in claim 18 in which the amount of the first metal is from about 0.01 to about 10 wt %, based on the total weight of the catalyst, and the amount of the second metal is from about 0.01 wt % to about 10 wt %, based on the total weight of the catalyst.

20. The fluidizable carbonylation catalyst as recited in claim 19 further comprising, optionally, at least one halogen promoter selected from iodine, bromine, and chlorine.

21. The fluidizable carbonylation catalyst as recited in claim 20 in which the halogen promoter is a metal halide.

22. The fluidizable carbonylation catalyst as recited in claim 21 in which the halogen promoter is sodium iodide, lithium iodide, or potassium iodide.

23. A fluidizable carbonylation catalyst prepared by a process comprising:
i) providing carbonized polysulfonated vinylaromatic polymer particles having
an average particle diameter of about 1 to about 200 μm;
a particle BET surface area of about 100 to about 2000 m²/g; and
a pore volume ratio of about 0.5 to about 20;
ii) contacting the particles in step (i) with a solution containing from about 0.01 wt % to about 20 wt %, based on the total weight of the solution, of at least one first metal selected from iron, cobalt, nickel, ruthenium, rhodium, palladium, osmium, iridium, platinum, and tin;
iii) drying the particles from step (ii);

24. The fluidizable carbonylation catalyst as recited in claim 23 further comprising:
iv) optionally, contacting the dried particles of step (iii) with a solution comprising from about 0.01 wt % to about 20 wt %, based on the total weight of the solution, of at least one second metal selected from alkali metals, alkaline earth metals, lanthanide metals, gold, mercury, vanadium, niobium, tantalum, titanium, zirconium, hafnium, molybdenum, tungsten, and rhenium;
v) drying the particles from step (iv);

25. The fluidizable carbonylation catalyst as recited in claim 24 further comprising the steps of:
vi) optionally, contacting the dried particles of step (iii) or step (v) with a solution comprising from about 0.01 wt % to about 20 wt %, based on the total weight of the solution, of a metal halide selected from sodium iodide, lithium iodide, or potassium iodide; and
vii) drying the particles from step (vi);

26. The fluidizable carbonylation catalyst as recited in claim 23 further comprising contacting the carbonized polysulfonated vinylaromatic polymer particles of step (i) with steam, oxygen, carbon dioxide, air, or ammonia at a temperature from about 700° C. to about 1000° C.

27. A process for the preparation of a carbonylation product comprising:
(1) feeding a gaseous mixture comprising carbon monoxide, a carbonylatable reactant, and a halide selected from chlorine, bromine, iodine and compounds thereof to a carbonylation zone which (i) contains a fluidizable carbonylation catalyst comprising carbonized polysulfonated vinylaromatic polymer particles and at least one first metal selected from iron, cobalt, nickel, ruthenium, rhodium, palladium, osmium, iridium, platinum, and tin in which the particles have a average particle diameter of about 1 to about 200 μm; (ii) is maintained under carbonylation conditions of temperature and pressure; and
(2) recovering a gaseous effluent comprising a carbonylation product from the carbonylation zone;
in which the gaseous mixture of step (1) is fed to the carbonylation zone at a superficial gas velocity sufficient to suspend the carbonylation catalyst in the gaseous mixture.

28. The process as recited in claim 27 in which the carbonylatable reactant comprises at least one compound selected from methanol, ethanol, methyl acetate, and dimethyl ether.

29. The process as recited in claim 28 in which the halide comprises at least one compound selected from iodine, hydrogen iodide and methyl iodide, and the carbonylation zone is maintained at a temperature of about 100 to 350° C. and a pressure of about 1 to 50 bar absolute.

30. The process as recited in claim 29 in which the first metal is rhodium or iridium.

31. The process as recited in claim 30 in which the carbonylation product comprises at least one compound selected from acetic acid, methyl acetate, and acetic anhydride.

32. The process as recited in claim 31 in which the fluidizable carbonylation catalyst further comprises at least one halogen promoter selected from iodine, bromine, and chlorine.

33. The process as recited in claim 32 in which the halogen promoter comprises at least one compound selected from sodium iodide, lithium iodide, and potassium iodide.

34. The process as recited in claim 33 in which the carbonylation catalyst further comprises, optionally, at least one second metal selected from alkali metals, alkaline earth metals, lanthanide metals, gold, mercury, vanadium, niobium, tantalum, titanium, zirconium, hafnium, molybdenum, tungsten, and rhenium.

35. The process as recited in claim 34 in which the amount of the first metal is from about 0.01 to about 10 wt %, based on the total weight of the catalyst, and the amount of the second metal is from about 0.01 wt % to about 10 wt %, based on the total weight of the catalyst.

36. A process for the preparation of acetic acid, methyl acetate, or a mixture thereof comprising:
(1) feeding a gaseous mixture comprising carbon monoxide, methanol, and a halide selected from iodine, hydrogen iodide, and methyl iodide to a carbonylation zone which (i) contains a fluidizable carbonylation catalyst comprising carbonized polysulfonated vinylaromatic polymer particles, rhodium, and lithium iodide in which the particles have an average particle diameter of about 1 to about 200 μm; (ii) is maintained at a temperature of about 150 to about 275° C. and a pressure of about 3 to about 50 bar absolute; and
(2) recovering a gaseous product comprising acetic acid from the carbonylation zone; and
in which the gaseous mixture of step (1) is fed to the carbonylation zone at a superficial gas velocity sufficient to suspend the carbonylation catalyst in the gaseous mixture.

37. The process as recited in claim 36 in which the fluidizable carbonylation catalyst has a BET surface area of about 500 to about 1200 m²/g; and a pore volume ratio of about 1.0 to about 8.

38. The process as recited in claim 37 the gaseous mixture contains water in an amount which gives a water:methanol mole ratio of about 0.01:1 to 1:1.

39. A process for the preparation of acetic acid, methyl acetate, or a mixture thereof comprising:
(1) feeding a gaseous mixture comprising carbon monoxide, methanol, and a halide selected from iodine, hydrogen iodide, or methyl iodide to a carbonylation zone which (i) contains the fluidizable carbonylation catalyst as recited in claim 16; (ii) is maintained at a temperature of about 150 to 275° C. and a pressure of about 3 to 50 bar absolute; and
(2) recovering a gaseous product comprising acetic acid from the carbonylation zone; and
in which the gaseous mixture of step (1) is fed to the carbonylation zone at a superficial gas velocity sufficient to suspend the carbonylation catalyst in the gaseous mixture.

40. A process for the preparation of a hydroformylation product comprising:
(1) feeding a gaseous mixture comprising carbon monoxide, hydrogen, and an olefin to a hydroformylation zone which (i) contains a fluidizable carbonylation catalyst comprising carbonized polysulfonated vinylaromatic polymer particles and at least one metal selected from iron, cobalt, nickel, ruthenium, rhodium, palladium, osmium, iridium, platinum, and tin in which the particles have a average particle diameter of about 1 to about 200 μm; (ii) is maintained under hydroformylation conditions of temperature and pressure; and
(2) recovering a gaseous effluent comprising a hydroformylation product from the hydroformylation zone;
in which the gaseous mixture of step (1) is fed to the hydroformylation zone at a superficial gas velocity sufficient to suspend the carbonylation catalyst in the gaseous mixture.
